(19) United States
(12) Patent Application Publication
(10) Pub. No.: US 2021/0068694 A1
(43) Pub. Date: Mar. 11, 2021

(54) SYSTEM FOR IDENTIFYING CARDIAC CONDUCTION PATTERNS

(71) Applicant: Acutus Medical, Inc., Carlsbad, CA (US)

(72) Inventors: Derrick Ren-yu CHOU, San Diego, CA (US); Graydon Ernest BEATTY, Carlsbad, CA (US); Nathan ANGEL, Oceanside, CA (US); R. Maxwell FLAHERTY, Topsfield, MA (US); J. Christopher FLAHERTY, Auburndale, FL (US)

(21) Appl. No.: 16/961,809

(22) PCT Filed: Jan. 22, 2019
(86) PCT No.: PCT/US2019/014498

§ 371 Date: Jul. 13, 2020

Related U.S. Application Data
(60) Provisional application No. 62/619,897, filed on Jan. 21, 2018.
(60) Provisional application No. 62/668,647, filed on May 8, 2018.

(51) Int. Cl. A61B 5/0468; A61B 5/042; A61B 5/00

(52) U.S. Cl. CPC A61B 5/0468; A61B 5/6858; A61B 5/042

(57) ABSTRACT

A system for diagnosing an arrhythmia of a patient comprises: a diagnostic catheter for insertion into the heart of the patient, the diagnostic catheter configured to record anatomic and electrical activity data of the patient; and a processing unit. The processing unit is configured to receive the recorded electrical activity data, and correlate the electrical activity data to the anatomic data. The processing unit comprises an algorithm configured to analyze the electrical activity at a location correlating to the anatomic data.

### RELATED APPLICATIONS

The present application claims priority under 35 USC 119(e) to U.S. Provisional Patent Application No. 62/619,897, entitled “System for Recognizing Cardiac Conduction Patterns,” filed Jan. 21, 2018, and U.S. Provisional Patent Application No. 62/668,647, entitled “System for Identifying Cardiac Conduction Patterns,” filed May 8, 2018, each of which is incorporated herein by reference in its entirety.

The present application, while not claiming priority to, may be related to U.S. Provisional Patent Application No. 62/757,961, entitled “Systems and Methods for Calculating Patient Information,” filed Nov. 9, 2018, which is hereby incorporated by reference.

The present application, while not claiming priority to, may be related to U.S. Provisional Patent Application No. 62/668,659, entitled “Cardiac Information Processing System,” filed May 8, 2018, which is hereby incorporated by reference.

The present application, while not claiming priority to, may be related to U.S. patent application Ser. No. 16/097,959, entitled “Cardiac Mapping System with Efficiency Algorithm,” filed Oct. 31, 2018, which is a 35 USC 371 national stage filing of Patent Cooperation Treaty Application No. PCT/US2017/030922, entitled “Cardiac Mapping System with Efficiency Algorithm”, filed May 3, 2017, which claimed priority to U.S. Provisional Patent Application No. 62/413,104, entitled “Cardiac Mapping System with Efficiency Algorithm,” filed Oct. 26, 2016 and U.S. Provisional Patent Application No. 62/331,364, entitled “Cardiac Mapping System with Efficiency Algorithm,” filed May 3, 2016, each of which is hereby incorporated by reference.

The present application, while not claiming priority to, may be related to U.S. patent application Ser. No. 16/097,955, entitled “Cardiac Information Dynamic Display System and Method,” filed Oct. 31, 2018, which is a 35 USC 371 national stage filing of Patent Cooperation Treaty Application No. PCT/US2017/030915, entitled “Cardiac Information Dynamic Display System and Method”, filed May 3, 2017, which claims priority to U.S. Provisional Patent Application No. 62/331,351, entitled “Cardiac Information Dynamic Display System and Method”, filed May 3, 2016, each of which is hereby incorporated by reference.

The present application, while not claiming priority to, may be related to Patent Cooperation Treaty Application No. PCT/US2017/056064, entitled “Ablation System with Force Control”, filed Oct. 11, 2017, which claims priority to U.S. Provisional Patent Application No. 62/406,748, entitled “Ablation System with Force Control”, filed Oct. 11, 2016, and U.S. Provisional Patent Application No. 62/504,139, entitled “Ablation System with Force Control”, filed May 20, 2017, each of which is hereby incorporated by reference.

The present application, while not claiming priority to, may be related to U.S. application Ser. No. 15/569,457, entitled “Localization System and Method Useful in the Acquisition and Analysis of Cardiac Information,” filed Oct. 26, 2017, which is a 35 USC 371 national stage filing of Patent Cooperation Treaty Application No. PCT/US2016/032420, entitled “Localization System and Method Useful in the Acquisition and Analysis of Cardiac Information”, filed May 13, 2016, which claims priority to U.S. Provisional Patent Application No. 62/161,213, entitled “Localization System and Method Useful in the Acquisition and Analysis of Cardiac Information”, filed May 13, 2015, each of which is hereby incorporated by reference.

The present application, while not claiming priority to, may be related to U.S. patent application Ser. No. 15/569,231, entitled “Cardiac Virtualization Test Tank and Testing System and Method,” filed Oct. 25, 2017, which is a 35 USC 371 national stage filing of Patent Cooperation Treaty Application No. PCT/US2016/031823, entitled “Cardiac Virtualization Test Tank and Testing System and Method”, filed May 11, 2016, which claims priority to U.S. Provisional Patent Application No. 62/160,501, entitled “Cardiac Virtualization Test Tank and Testing System and Method”, filed May 12, 2015, each of which is hereby incorporated by reference.

The present application, while not claiming priority to, may be related to U.S. application Ser. No. 15/569,185, entitled “Ultrasound Sequencing System and Method,” filed Oct. 25, 2017, which is a 35 USC 371 national stage filing of Patent Cooperation Treaty Application No. PCT/US2016/032017, entitled “Ultrasound Sequencing System and Method”, filed May 12, 2016, which claims priority to U.S. Provisional Patent Application No. 62/160,529, entitled “Ultrasound Sequencing System and Method”, filed May 12, 2015, each of which is hereby incorporated by reference.

The present application, while not claiming priority to, may be related to U.S. application Ser. No. 14/916,056, entitled “Devices and Methods for Determination of Electrical Dipole Densities on a Cardiac Surface”, filed Sep. 10, 2014, which is a 35 USC 371 national stage filing of Patent Cooperation Treaty Application No. PCT/US2014/54942, entitled “Devices and Methods for Determination of Electrical Dipole Densities on a Cardiac Surface”, filed Sep. 10, 2014, which claims priority to U.S. Provisional Patent Application No. 61/877,617, entitled “Devices and Methods for Determination of Electrical Dipole Densities on a Cardiac Surface”, filed Sep. 13, 2013, each of which is hereby incorporated by reference.

The present application, while not claiming priority to, may be related to U.S. application Ser. No. 15/128,563, entitled “Cardiac Analysis User Interface System and Method”, filed Sep. 23, 2016, which is a 35 USC 371 national stage filing of Patent Cooperation Treaty Application No. PCT/US2015/22187, entitled “Cardiac Analysis User Interface System and Method”, filed Mar. 24, 2015, which claims priority to U.S. Patent Provisional Application No. 61/970,027, entitled “Cardiac Analysis User Interface System and Method”, filed Mar. 28, 2014, each of which is hereby incorporated by reference.

The present application, while not claiming priority to, may be related to U.S. application Ser. No. 16/111,538, entitled “Gas-Elimination Patient Access Device”, filed Aug. 24, 2018, which is a continuation of U.S. Pat. No. 10,071,227, entitled “Gas-Elimination Patient Access Device”, filed Jan. 14, 2015, which was a 35 USC 371 national stage filing of Patent Cooperation Treaty Application No. PCT/US2015/011312, entitled “Gas-Elimination Patient Access Device”, filed Jan. 14, 2015, which claimed priority to U.S. Provisional Patent Application No. 61/928,704, entitled “Gas-Elimination Patient Access Device”, filed Jan. 17, 2014, each of which is hereby incorporated by reference.

The present application, while not claiming priority to, may be related to U.S. patent application Ser. No. 16/242,810, entitled “Expandable Catheter Assembly with Flexible Printed Circuit Board (PCB) Electrical Pathways”, filed Jan. 8, 2019, which is a continuation of patent application Ser. No. 14/762,944, entitled “Expandable Catheter Assembly with Flexible Printed Circuit Board (PCB) Electrical Pathways”, filed Jul. 23, 2015, which was a 35 USC 371 national stage filing of Patent Cooperation Treaty Application No. PCT/US2014/15261, entitled “Expandable Catheter Assembly with Flexible Printed Circuit Board (PCB) Electrical Pathways”, filed Feb. 7, 2014, which claims priority to U.S. Provisional Patent Application Ser. No. 61/762,363, entitled “Expandable Catheter Assembly with Flexible Printed Circuit Board (PCB) Electrical Pathways”, filed Feb. 8, 2013, which is hereby incorporated by reference.

The present application, while not claiming priority to, may be related to U.S. patent application Ser. No. 16/012,051, entitled “Catheter, System and Methods of Medical Uses of Same, Including Diagnostic and Treatment Uses for the Heart,” filed Jun. 19, 2018, which is a continuation of U.S. Pat. No. 10,004,459, entitled “Catheter, System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, published as WO 2014/036439, which claims priority to U.S. Provisional Patent Application No. 61/695,535, entitled “System and Method for Diagnosing and Treating Heart Tissue”, filed Aug. 31, 2012, each of which is hereby incorporated by reference.

The present application, while not claiming priority to, may be related to U.S. Design patent application No. 29/593,043, entitled “Set of Transducer-Electrode Pairs for a Catheter,” filed Feb. 6, 2017, which is a divisional of U.S. Design patent No. D782686, entitled “Transducer Electrode Arrangement”, filed Dec. 2, 2013, which is a continuation-in-part of Patent Cooperation Treaty Application No. PCT/US2013/057579, entitled “Catheter System and Methods of Medical Uses of Same, Including Diagnostic and Treatment Uses for the Heart”, filed Aug. 30, 2013, which is hereby incorporated by reference.

The present application, while not claiming priority to, may be related to U.S. patent application Ser. No. 15/926,187, entitled “Device and Method for the Geometric Determination of Electrical Dipole Densities on the Cardiac Wall,” filed Mar. 20, 2018, which is a continuation of U.S. Pat. No. 9,968,268, entitled “Device and Method for the Geometric Determination of Electrical Dipole Densities on the Cardiac Wall,” which is a continuation of U.S. Pat. No. 9,757,044, entitled “Device and Method for the Geometric Determination of Electrical Dipole Densities on the Cardiac Wall”, which is a 35 USC 371 national stage filing of Patent Cooperation Treaty Application No. PCT/US2012/028593, entitled “Device and Method for the Geometric Determination of Electrical Dipole Densities on the Cardiac Wall,” filed Mar. 9, 2012, which claimed priority to U.S. Provisional Patent Application No. 61/451,357, entitled “Device and Method for the Geometric Determination of Electrical Dipole Densities on the Cardiac Wall,” filed Mar. 10, 2011, each of which is hereby incorporated by reference.

The present application, while not claiming priority to, may be related to U.S. patent application Ser. No. 15/882,097, entitled “Device and Method for the Geometric Determination of Electrical Dipole Densities on the Cardiac Wall,” filed Jan. 29, 2018, which is a continuation of U.S. Pat. No. 9,913,589, entitled “Device and Method for the Geometric Determination of Electrical Dipole Densities on the Cardiac Wall”, filed Oct. 25, 2016, which is a continuation of U.S. Pat. No. 9,504,395, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, issued Aug. 20, 2013, which was a 35 USC 371 national stage application of Patent Cooperation Treaty Application No. PCT/IB09/00071 filed Jan. 16, 2009, entitled “A Device and Method for the Geometric Determination of Electrical Dipole Densities on the Cardiac Wall”, which claimed priority to Swiss Patent Application No. 00068/08 filed Jan. 17, 2008, each of which is hereby incorporated by reference.

The present application, while not claiming priority to, may be related to U.S. patent application Ser. No. 16/014,370, entitled “Method and Device for Determining and Presenting Surface Charge and Dipole Densities on Cardiac Walls,” filed Jun. 21, 2018, which is a continuation of U.S. patent application Ser. No. 15/435,763, entitled “Method and Device for Determining and Presenting Surface Charge and Dipole Densities on Cardiac Walls,” filed Feb. 17, 2017, which is a continuation of U.S. Pat. No. 9,610,024, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158, entitled “Method and Device for Determining and Presenting Surface Charge and Dipole Densities on Cardiac Walls”, issued Dec. 23, 2014, which is a continuation of U.S. Pat. No. 8,700,119, entitled “Method and Device for Determining and Presenting Surface Charge and Dipole Densities on Cardiac Walls”, issued Apr. 15, 2014, which is a continuation of U.S. Pat. No. 8,417,313, entitled “Method and Device for Determining and Presenting Surface Charge and Dipole Densities on Cardiac Walls”, issued Apr. 9, 2013, which was a 35 USC 371 national stage filing of PCT Application No. CH2007/000380, entitled “Method and Device for Determining and Presenting Surface Charge and Dipole Densities on Cardiac Walls”, filed Aug. 3, 2007, which claimed priority to Swiss Patent Application No. 1251/06 filed Aug. 3, 2006, each of which is hereby incorporated by reference.

### FIELD OF THE INVENTION

The present invention is generally related to systems and methods that may be useful for the diagnosis and treatment of cardiac arrhythmias or other abnormalities, in particular, the present invention is related to systems, devices, and methods useful in displaying cardiac activities associated with diagnosing and treating such arrhythmias or other abnormalities.

### BACKGROUND

Cardiac signals (e.g. charge density, dipole density, voltage, etc.) vary across the endocardial surface in magnitude. The magnitude of these signals is dependent on several factors, including local tissue characteristics (e.g. healthy vs. disease/scar/fibrosis/lesion) and regional activation characteristics (e.g. “electrical mass” of activated tissue prior to activation of the local cells). A common practice is to assign a single threshold for all signals at all times across the surface. The use of a single threshold can cause low-amplitude activation to be missed or cause high-amplitude activation to dominate/saturate, leading to confusion in interpretation of the map. Failure to properly detect activation can lead to imprecise identification of regions of interest for therapy delivery or incomplete characterization of ablation efficacy (excess or lack of block).

The continuous, global mapping of atrial fibrillation yields a tremendous volume of temporally- and spatially-variable activation patterns. A limited, discrete sampling of map data may be insufficient to provide a comprehensive picture of the drivers, mechanisms, and supporting substrate for the arrhythmia. Clinician review of long durations of AF can be challenging to remember and piece together to complete the “bigger picture.”

For these and other reasons, there is a general need to algorithmically provide an objective analysis of conduction patterns.

### SUMMARY

Embodiments of the systems, devices and methods described herein can be directed to systems, devices and methods for diagnosing an arrhythmia of a patient.

According to an aspect of the present inventive concepts, a system for diagnosing an arrhythmia of a patient comprises: a diagnostic catheter for insertion into the heart of the patient, and a processing unit. The diagnostic catheter is configured to record anatomic and electrical activity data of the patient. The processing unit is configured to receive the recorded electrical activity data, and correlate the electrical activity data to the anatomic data. The processing unit comprises an algorithm configured to determine the conduction velocity of a depolarizing conduction wave at a location correlating to the anatomic data.

According to an aspect of the present inventive concepts, a system for diagnosing an arrhythmia of a patient comprises: a diagnostic catheter for insertion into the heart of the patient, and a processing unit. The diagnostic catheter is configured to record anatomic and electrical activity data of the patient. The processing unit is configured to receive the recorded electrical activity data, and correlate the electrical activity data to the anatomic data. The processing unit comprises an algorithm configured to identify rotational conduction at a location correlating to the anatomic data.

According to an aspect of the present inventive concepts, a system for diagnosing an arrhythmia of a patient comprises: a diagnostic catheter for insertion into the heart of the patient, and a processing unit. The diagnostic catheter is configured to record anatomic and electrical activity data of the patient. The processing unit is configured to receive the recorded electrical activity data, and correlate the electrical activity data to the anatomic data. The processing unit comprises an algorithm configured to identify irregular conduction at a location correlating to the anatomic data.

According to an aspect of the present inventive concepts, a system for diagnosing an arrhythmia of a patient comprises: a diagnostic catheter for insertion into the heart of the patient, and a processing unit. The diagnostic catheter is configured to record anatomic and electrical activity data of the patient. The processing unit is configured to receive the recorded electrical activity data, and correlate the electrical activity data to the anatomic data. The processing unit comprises an algorithm configured to identify focal activation at a location correlating to the anatomic data.

According to an aspect of the present inventive concepts, a system for producing diagnostic results related to a cardiac condition of a patient, comprises: a diagnostic catheter for insertion into the heart of the patient, the diagnostic catheter configured to record electrical activity data of the patient at multiple recording locations; and a processing unit for receiving the recorded electrical activity data. The system further comprises an algorithm configured to perform a complexity assessment using the recorded electrical activity data and produce the diagnostic results based on the complexity assessment.

In some embodiments, the diagnostic results comprise an assessment of complexity or an assessment of a variation of complexity over time and/or space. The diagnostic results can comprise a variation of complexity over time and space.

In some embodiments, the complexity assessment comprises a macro-level complexity assessment.

In some embodiments, the complexity assessment represents an assessment of a portion of a heart chamber, and the multiple recording locations comprise at least three recording locations within a heart chamber, and the system determines calculated electrical activity data for at least three vertices on the heart wall, and the calculation is based on electrical activity data recorded at the at least three recording locations. The at least three recording locations can comprise at least three locations on the heart wall. The portion of the heart chamber can comprise no more than 7 cm2, no more than 4 cm2, and/or no more than 1 cm2 of surface of the heart wall. The at least three recording locations can comprise at least one location offset from the heart wall.

In some embodiments, the complexity assessment represents an assessment of a portion of a heart chamber, and the multiple recording locations comprise at least 24 recording locations within a heart chamber, and the system determines calculated electrical activity data for at least 64 vertices on the heart wall, and the calculation is based on electrical activity data recorded at the at least 24 recording locations. The at least 24 recording locations can comprise at least 24 heart wall locations. The at least 24 recording locations can comprise at least 48 heart wall locations. The at least 24 recording locations can comprise at least 48 heart wall locations. The at least 24 recording locations can comprise at least 48 locations within the heart chamber. The at least 24 recording locations can comprise at least 64 locations within the heart chamber. The at least 64 vertices can comprise at least 100 vertices. The at least 64 vertices can comprise at least 500 vertices. The at least 64 vertices can comprise at least 3000 vertices. The at least 64 vertices can comprise at least 5000 vertices. The portion of the heart chamber can comprise at least 1 cm2, at least 4 cm2, and/or at least 7 cm2 of surface of the heart wall. The portion of the heart chamber can comprise a portion of an atria of the heart.

In some embodiments, the system determines calculated electrical activity data for multiple vertices on the heart wall, and the calculation is based on electrical activity data recorded at the at least three recording locations. The recorded electrical activity data can comprise voltage data recorded at multiple locations within a chamber of the patient's heart, and the multiple locations can include at least one location offset from the heart wall. The recorded electrical activity data can comprise voltage data recorded at multiple locations within a chamber of the patient's heart, and the multiple locations can include at least one location on the heart wall. The recorded electrical activity data can comprise voltage data recorded at multiple locations within a chamber of the patient's heart, and the multiple locations can include at least one location on the heart wall and at least one location offset from the heart wall. The processing unit can further comprise a second algorithm, and the recorded electrical activity data can comprise recorded voltage data, and the second algorithm can be configured to calculate surface charge data and/or dipole density data for each of the multiple vertices based on the recorded voltage data, and the complexity assessment can be based on the surface charge data and/or the dipole density data. The processing unit can further comprise a third algorithm, and the third algorithm can be configured to convert the surface charge data and/or dipole density data into surface voltage data, and the complexity assessment can be based on the surface voltage data.

In some embodiments, the complexity assessment is based on electrical activity data comprising between 1 and 10 activations.

In some embodiments, the complexity assessment is based on electrical activity data recorded over a time period between 0.3 ms and 2000 ms. The complexity assessment can be based on electrical activity data recorded over a time period of approximately 150 ms.

In some embodiments, the complexity assessment is based on electrical activity data comprising between 3 and 3000 activations. The complexity assessment can be based on electrical activity data comprising between 10 and 600 activations. The complexity assessment can be based on electrical activity data comprising between 25 and 300 activations.

In some embodiments, the complexity assessment is based on electrical activity data recorded over a time period between 0.3 secs and 500 secs. The complexity assessment can be based on electrical activity data recorded over a time period between 1 sec and 90 secs. The complexity assessment can be based on electrical activity data recorded over a time period between 4 secs and 30 secs.

In some embodiments, the complexity assessment is based on electrical activity data comprising between 2,000 and 300,000 activations. The complexity assessment can be based on electrical activity data comprising between 6,000 and 40,000 activations.

In some embodiments, the complexity assessment is based on electrical activity data recorded over a time period between 5 mins and 8 hrs. The complexity assessment can be based on electrical activity data recorded over a time period between 15 mins and 50 mins.

In some embodiments, the diagnostic results comprise an assessment of complexity at a single heart wall location. The system can further comprise a display, and the system can provide on the display the diagnostic results relative to an image of the patient's anatomy.

In some embodiments, the diagnostic results comprise an assessment of complexity at multiple heart wall locations. The system can further comprise a display, and the system can provide on the display the diagnostic results relative to an image of the patient's anatomy.

In some embodiments, the diagnostic results comprise an assessment of complexity over time. The diagnostic results can comprise an assessment of complexity over a pre-determined time duration.

In some embodiments, the diagnostic catheter comprises at least one electrode.

In some embodiments, the diagnostic catheter comprises at least three electrodes.

In some embodiments, the diagnostic catheter comprises at least one ultrasound transducer.

In some embodiments, the diagnostic catheter comprises multiple splines, and each spline comprises at least one electrode and at least one ultrasound transducer.

In some embodiments, the cardiac condition comprises an arrhythmia. The cardiac condition can comprise atrial fibrillation.

In some embodiments, the cardiac condition comprises a condition selected from the group consisting of: atrial fibrillation; atrial flutter; atrial tachycardia; atrial bradycardia, ventricular tachycardia; ventricular bradycardia; ectopy; congestive heart failure; angina; arterial stenosis; and combinations thereof.

In some embodiments, the cardiac condition comprises a condition selected from the group consisting of: heterogeneous activation, conduction, depolarization, and/or repolarization that varies in time, space, magnitude, and/or state; irregular patterns such as focal, re-entrant, rotational, pivoting, irregular in direction, irregular in velocity; functional block; permanent block; and combinations thereof.

In some embodiments, the system is further configured to collect additional patient data, and the complexity assessment is further based on the additional patient data. The diagnostic catheter can be configured to record the additional patient data. The diagnostic catheter can comprise at least one sensor configured to record the additional patient data. The system can comprise at least one sensor configured to record the additional patient data. The at least one sensor can be configured to be inserted in the patient when recording the additional patient data. The at least one sensor can be configured to be positioned external to the patient when recording the additional patient data. The sensor can comprise a sensor selected from the group consisting of: an electrode or other sensor for recording electrical activity; a force sensor; a pressure sensor; a magnetic sensor; a motion sensor; a velocity sensor; an accelerometer; a strain gauge; a physiologic sensor; a glucose sensor; a pH sensor; a blood sensor; a blood gas sensor; a blood pressure sensor; a flow sensor; an optical sensor; a spectrometer; an interferometer; a measuring sensor, such as to measure size, distance, and/or thickness; a tissue assessment sensor; and combinations thereof. The additional patient data can comprise: mechanical information; physiologic information, and/or functional information of the patient. The additional patient data can comprise data related to a parameter selected from the group consisting of: heart wall motion; heart wall velocity; heart tissue strain; magnitude and/or direction of heart blood flow; vorticity of blood; heart valve mechanics; blood pressure; tissue properties, such as density, tissue characteristics and/or biomarkers for tissue characteristics, such as metabolic activity or pharmaceutical uptake; tissue composition (e.g. collagen, myocardium, fat, connective tissue); and combinations thereof. The complexity assessment can include an assessment of a characteristic selected from the group consisting of: electrical-mechanical delay of tissue; magnitude ratio of an electrical to a mechanical characteristic; and combinations thereof.

In some embodiments, the system is further configured to treat an arrhythmia, and the system further comprises an ablation catheter for insertion into the heart of the patient, and the ablation catheter is configured to deliver ablation energy to various locations on the heart wall. The algorithm can be configured to determine at least one ablation location, the at least one ablation location can comprise one or more heart wall locations for receiving the ablation energy from the ablation catheter, the at least one ablation location can be determined based on the complexity assessment and/or the diagnostic results. The at least one ablation location can comprise one or more heart locations where complexity exceeds a threshold. The at least one ablation location can comprise a location of highest complexity in a region of multiple determined complexities. The ablation catheter can be configured to deliver one or more ablation energies selected from the group consisting of: electromagnetic energy; RF energy; microwave energy; thermal energy; heat energy; cryogenic energy; light energy; laser light energy; chemical energy; sound energy; ultrasound energy; mechanical energy; and combinations thereof. The system can further comprise an energy delivery unit configured to provide the ablation energy to the ablation catheter. The energy delivery unit can be configured to deliver one or more ablation energies selected from the group consisting of: electromagnetic energy; RF energy; microwave energy; thermal energy; heat energy; cryogenic energy; light energy; laser light energy; chemical energy; sound energy; ultrasound energy; and combinations thereof.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a block diagram of a cardiac information processing system, consistent with the present inventive concepts.

FIG. 2A illustrates a visual representation of a data structure of a cardiac information processing system, consistent with the present inventive concepts.

FIG. 2B illustrates a visual representation of a portion of a data structure of a cardiac information processing system, consistent with the present inventive concepts.

FIG. 3 illustrates a schematic view of an algorithm for performing a complexity assessment, consistent with the present inventive concepts.

FIG. 3A illustrates a schematic view of an algorithm for performing a complexity assessment, consistent with the present inventive concepts.

FIG. 4 illustrates a schematic view of an algorithm for determining conduction velocity data, consistent with the present inventive concepts.

FIG. 5 illustrates a schematic view of an algorithm for determining localized rotational activity, consistent with the present inventive concepts.

FIG. 5A illustrates a graphical representation of anatomic data including a neighborhood of vertices defined by an outer ring of vertices, consistent with the present inventive concepts.

FIG. 5B illustrates a simplified representation of a neighborhood including an outer ring of vertices positioned about a central vertex, consistent with the present inventive concepts.

FIG. 5C illustrates a representative anatomy showing a propagating wave rotating about a neighborhood, consistent with the present inventive concepts.

FIG. 5D illustrates a plot of activation times in the outer ring of vertices of FIG. 5C, consistent with the present inventive concepts.

FIG. 5E illustrates a graph of conduction velocity vectors of FIG. 5C, consistent with the present inventive concepts.

FIG. 6 illustrates a schematic view of an algorithm for determining localized irregular activity, consistent with the present inventive concepts.

FIG. 6A illustrates an example of a propagation wave showing irregular activity, consistent with the present inventive concepts.

FIG. 7 illustrates a schematic view of an algorithm for determining focal activation, consistent with the present inventive concepts.

FIGS. 7A and 7B illustrate a representative anatomy showing focal activation, consistent with the present inventive concepts.

FIG. 8 illustrates a display on which cardiac data can be rendered, consistent with the present inventive concepts.

FIGS. 9 and 9A illustrate a schematic view of a mapping catheter, and a perspective anatomic view of a heart chamber with a mapping catheter inserted into the chamber, consistent with the present inventive concepts

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values and/or outside a threshold range of values, to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values. Thresholds can be defined by a user (e.g. a clinician of the patient), and/or system defined (e.g. in manufacturing of the system).

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to record data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are cardiac information systems for producing diagnostic results related to a cardiac condition of a patient. The systems can be used to perform a medical procedure on a patient, such as a diagnostic, prognostic, and/or therapeutic procedure on the patient. The systems can identify cardiac conduction patterns of a patient, such as an arrhythmia patient. The system includes a diagnostic catheter for insertion into the heart of the patient. The diagnostic catheter can be configured to record electrical activity data of the patient, such as when the catheter includes one or more electrodes for measuring voltage. The system can further include a processing unit for receiving the recorded electrical activity data. The processing unit can comprise an algorithm configured to perform one or more functions, such as to produce calculated electrical activity data, complexity assessment, and/or the diagnostic results. In some embodiments, the algorithm performs a complexity assessment to produce the diagnostic results. In some embodiments, the complexity assessment is performed by one or more algorithms described herein, which solely or in combination with another algorithm perform a complexity assessment. In some embodiments, the system further includes a treatment device, such as a cardiac ablation device and/or a pharmaceutical agent.

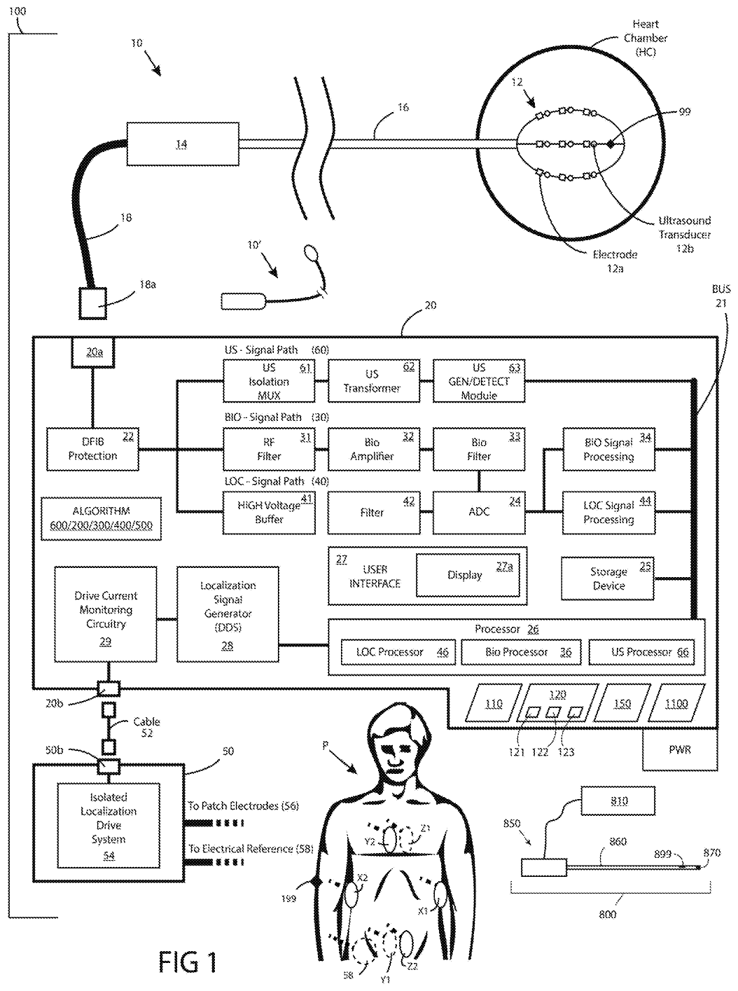

Referring now to FIG. 1, a block diagram of an embodiment of a cardiac information processing system is illustrated, consistent with the present inventive concepts. The cardiac information processing system, system 100 shown, can be or include a system configured to perform cardiac mapping, diagnosis, prognosis, and/or treatment, such as for treating a disease or disorder of a patient, such as an arrhythmia or other cardiac condition as described herein. Additionally or alternatively, system 100 can be a system configured for teaching and or validating devices and methods of diagnosing and/or treating cardiac abnormalities or disease of a patient P. System 100 can further be used for generating displays of cardiac activity, such as dynamic displays of active wave fronts propagating across surfaces of the heart. In some embodiments, system 100 produces diagnostic results 1100. Diagnostic results 1100 represent diagnostic data related to a cardiac condition of a patient, such as diagnostic results based on a complexity assessment as described herein.

System 100 includes a catheter 10, a cardiac information console 20, and a patient interface module 50 that can be configured to cooperate (e.g. collectively cooperate) to accomplish the various functions of the system 100. System 100 can include a single power supply (PWR), which can be shared by console 20 and the patient interface module 50. Use of a single power supply in this way can greatly reduce the chance for leakage currents to propagate into the patient interface module 50 and cause errors in localization (e.g. the process of determining the location of one or more electrodes within the body of patient P). Console 20 includes bus 21 which electrically and/or otherwise operatively connects various components of console 20 to each other, as shown in FIG. 1.

Catheter 10 includes an electrode array 12 that can be percutaneously delivered to a heart chamber (HC). In this embodiment, the array of electrodes 12 has a known spatial configuration in three-dimensional (3D) space. For example, in an expanded state the physical relationship of the electrode array 12 can be known or reliably assumed. Electrode array 12 can include at least one electrode 12a, or at least three electrodes 12a. Diagnostic catheter 10 also includes a handle 14, and an elongate flexible shaft 16 extending from handle 14. Attached to a distal end of shaft 16 is the electrode array 12, such as a radially expandable and/or compactable assembly. In this embodiment, the electrode array 12 is shown as a basket array, but the electrode array 12 could take other forms in other embodiments. In some embodiments, expandable electrode array 12 can be constructed and arranged as described in reference to applicant's International PCT Patent Application Serial Number PCT/US2013/057579, titled “SYSTEM AND METHOD FOR DIAGNOSING AND TREATING HEART TISSUE,” filed Aug. 30, 2013, and International PCT Patent Application Serial Number PCT/US2014/015261, titled “EXPANDABLE CATHETER ASSEMBLY WITH FLEXIBLE PRINTED CIRCUIT BOARD,” filed Feb. 7, 2014, the content of each of which is incorporated herein by reference in its entirety for all purposes. In other embodiments, expandable electrode array 12 can comprise a balloon, radially deployable arms, spiral array, and/or other expandable and compactible structure (e.g. a resiliently biased structure).

Shaft 16 and expandable electrode array 12 are constructed and arranged to be inserted into a body (e.g. an animal body or a human body, such as the body of Patient P), and advanced through a body vessel, such as a femoral vein and/or other blood vessel. Shaft 16 and electrode array 12 can be constructed and arranged to be inserted through an introducer (not shown, but such as a transseptal sheath), such as when electrode array 12 is in a compacted state, and slidingly advanced through a lumen of the introducer into a body space, such as a chamber of the heart (HC), such as the right atrium or the left atrium, as examples.

Expandable electrode array 12 can comprise multiple splines (e.g. multiple splines resiliently biased in the basket shape shown in FIG. 1), each spline having a plurality of electrodes 12a and/or a plurality of ultrasound (US) transducers 12b. Three splines are visible in FIG. 1, but the basket array is not limited to three splines, more or less splines can be included in the basket array. Each electrode 12a can be configured to record (e.g. record, measure, and/or sense, herein) a bio-potential (also referred to as “electrical activity” herein), such as the voltage level at a location on a surface of the heart and/or at a location within a heart chamber HC. Recorded electrical activity is stored by system 100 as electrical activity data 120a. System 100 can perform one or more calculations on the recorded electrical activity data 120a to produce calculated electrical activity data 120b. Electrical activity data 120 can comprise recorded electrical activity data 120a and/or calculated electrical activity data 120b. Calculated electrical activity data 120b can comprise data selected from the group consisting of: voltage data; mathematically processed voltage data (e.g. data that is averaged, integrated, sorted, had minimum and/or maximum values determined, and/or otherwise is mathematically processed); surface charge data; dipole density data; timing data of electrical events; filtered electrical data; electrical pattern and/or template data; an image formed by electrical values at multiple locations; and combinations of one, two, or more of these. As used herein, the term dipole density, surface charge, and surface charge density, shall be used interchangeably.

Calculated electrical activity data 120b can comprise data that represents instances of electrical activation (also referred to as “activation” herein) of heart tissue, activation timing data 121. In some embodiments, calculated electrical activity data 120b comprises data that represents, conduction velocity, conduction velocity data 122, and/or conduction divergence, conduction divergence data 123, each described herebelow. Calculated electrical activity data 120b can be correlated to one or more locations of the heart, referred to as a vertex (single location) and vertices (multiple locations) herein. In some embodiments, calculated electrical activity data comprises data selected from the group consisting of: electrical differences (e.g. deltas); averages; weighted averages; patterns and/or templates; degree-of-fit (e.g. best-fit) to one or more patterns or templates; “flow” between two or more images formed by electrical values at multiple locations (e.g. as calculated by one, two, or more optical flow algorithms, such as Horn-Schunck and/or a Lucas-Kanade algorithm); data analytics and/or statistics techniques, such as classification or categorization, of electrical activity using a training data set (e.g. separately acquired data, such as historical data); a computationally-optimized fit (e.g. machine learning or predictive analysis, such as by neural network or deep learning, cluster analysis); and combinations of one, two, or more of these. The calculated electrical activity data can comprise a probabilistic model that uses one or more of the aforementioned methods as inputs.

In some embodiments, activation is determined by an algorithm (e.g. an activation detection algorithm) which can include: comparing electrical data to a threshold; measurement of the slope and/or maximum and/or minimum of the electrical data; comparing electrical data at one location to electrical data at one or more nearby locations (e.g. weighted comparison); and combinations of these. In some embodiments, the activation detection algorithm can be of similar construction and arrangement as described in reference to applicant's International PCT Patent Application Serial Number PCT/US2017/030915, titled “CARDIAC INFORMATION DYNAMIC DISPLAY SYSTEM AND METHOD”, filed May 3, 2017, and International PCT Patent Application Serial Number PCT/US2017/030922, titled “CARDIAC MAPPING SYSTEM WITH EFFICIENCY ALGORITHM”, filed May 3, 2017, the content of each of which is incorporated herein by reference in its entirety for all purposes. To promote the spatial continuity for a propagation history map, the activation detection algorithm can comprise two parallel lines considering both raw signal (e.g. dipole density data and/or voltage data) together with a spatial Laplacian signal. In some embodiments, the activation detection algorithm further includes conduction velocity as one consideration of selecting between potential active timings, as well as developing voting schemes on multiple features, such as gradient, spatial Laplacian, peak amplitude, and/or other such features.

Expanding upon the conduction velocity addition to the activation detection, the problem can be represented as a cost function with either regularization on the conduction velocity or as an inequality constraint on the conduction velocity. In some embodiments, the activation detection algorithm creates a Gaussian probability distribution function around each detected activation where the highest probability is at the currently detected activation. Given no constraints, maximizing the probability of activation for every channel can output a propagation history. Alternatively, including at least one constraint can limit the solution to comprise a physiologically reasonable conduction (e.g., less than 2 m/s) and can be configured to shift the activations slightly from the currently selected activation times. Below shows an example of how the cost function can be written with constrained conduction velocity:

$$max ( {\sum }_{i = 1}^{\# of Vert} P ( i , {τ}_{i} ) ) , s . t . Conduction {Velocity}_{i} < Constant \qquad ( 1 )$$

where P is the probability of activation occurring at a particular vertex, i, at time, τ. The conduction velocity calculation is dependent on τ.

In some embodiments, the activation detection algorithm comprises a local minimum of temporal derivative of unipolar electrogram with a minimum separation between activations set to a time threshold (e.g. between 50-150 ms)

In some embodiments, the activation detection algorithm comprises a local minimum or maximum of bipolar or Laplacian electrograms with a minimum separation between activations set to a time threshold (e.g. between 50-150 ms)

In some embodiments, the activation detection algorithm comprises standard filtering with a bandpass of (0.5 to 1 Hz)-(100-300 Hz), or after an aggressive band pass of (10-30 Hz)-(100-300 Hz).

In some embodiments, the activation detection algorithm comprises a local minimum and/or maximum of temporal derivative of bipolar electrograms or Laplacian electrograms with a minimum separation between activations set to a time threshold (e.g. between 50-150 ms). The activation detection algorithm can further comprise standard filtering with a bandpass of (0.5 to 1 Hz)-(100-300 Hz) or after aggressive band pass of (10-30 Hz)-(100-300 Hz).

In some embodiments, the activation detection algorithm comprises zero crossings of Laplacian electrograms after a negative deflection with a minimum separation between activations set to a time threshold (e.g. between 50-150 ms).

In some embodiments, the activation detection algorithm comprises local maximums of Hilbert transformed electrograms (Phase Mapping) with a minimum separation between activations set to a time threshold (e.g. between 50-150 ms).

In some embodiments, the activation detection algorithm can comprise an algorithm expressed as a supervised learning problem utilizing machine learning (e.g. neural networks, support vector machines, and/or deep learning). In these embodiments, the algorithm can use a training data set, such as a data set including historic data and/or simulated data.

Each US transducer 12b can be configured to transmit an ultrasound signal and receive ultrasound reflections to determine the range to a reflecting target such as at a point on the surface of a heart chamber (H), to provide anatomic data used in a digital model creation of the anatomy. Recorded ultrasound data and/or other anatomic data can be stored by system 100 as anatomic data 110. Electrical activity data 120 (e.g. including activation timing data 121, conduction velocity data 122, and/or conduction divergence data 123) and/or anatomic data 110 can be stored in memory of system 100, for example storage device 25 described herebelow.

As a non-limiting example, three electrodes 12a and three US transducers 12b are shown on each spline in this embodiment. However, in other embodiments, the basket array can include more or less electrodes and/or more or less US transducers. Furthermore, the electrodes 12a and transducers 12b can be arranged in pairs. Here, one electrode 12a is paired with one transducer 12b, with multiple electrode-transducer pairs per spline. The inventive concept is not, however, limited to this particular electrode-transducer arrangement. In other embodiments, not all electrodes 12a and transducers 12b need to be arranged in pairs, some could be arranged in pairs while others are not arranged in pairs. Also, in some embodiments, not all splines comprise the same arrangement of electrodes 12a and transducers 12b. Additionally, in some embodiments, electrodes 12a are arranged on a first set of splines, while transducers 12b are arranged on a second set of splines. Array 12 can comprise at least four electrodes 12a, such as at least 24 electrodes 12a, such as at least 48 electrodes. Array 12 can comprise at least three splines, such as at least four splines, such as at least six splines.

In some embodiments, a second catheter, catheter 10′, is used in conjunction with catheter 10, for example a basket or other array of electrodes of catheter 10′ can be positioned in a separate heart chamber to simultaneously map more than one chamber of the heart. Catheter 10′ can be of similar or dissimilar construction to catheter 10, described herein. The electrode array of catheter 10′ can be arranged in a different configuration than the electrode array 12 of catheter 10. For example, the array of catheter 10′ can only have 24 electrodes and no US transducers while array 12 of catheter 10 possesses 48 electrodes and 48 US transducers. Catheter 10 and/or 10′ can comprise two or more electrode arrays, such as array 12 shown, and a second array, positioned proximal to array 12 (e.g. on shaft 16 of catheter 10 or 10′).

Catheter 10 can comprise a cable or other conduit, such as cable 18, configured to electrically, optically, and/or electro-optically connect catheter 10 to console 20 via connectors 18a and 20a, respectively. In some embodiments, cable 18 comprises a mechanism selected from the group consisting of: a cable such as a steering cable; a mechanical linkage; a hydraulic tube; a pneumatic tube; and combinations of one or more of these.

Patient interface module 50 can be configured to electrically isolate one or more components of console 20 from patient P (e.g. to prevent undesired delivery of a shock or other undesired electrical energy to patient P). The patient interface module 50 can be integral with console 20 and/or it can comprise a separate discrete component (e.g. separate housing), as is shown. Console 20 comprises one or more connectors 20b, each comprising a jack, plug, terminal, port, or other custom or standard electrical, optical, and/or mechanical connector. In some embodiments, the connectors 20b are terminated to maintain desirable input impedance over RF frequencies, such as 10 kilohertz to 20 megahertz. In some embodiments, the termination is achieved by terminating the cable shield with a filter. In some embodiments, the terminating filters provide high input impedance in one frequency range, for example to minimize leakage at localization frequencies, and low input impedance in a different frequency range, for example to achieve maximum signal integrity at ultrasound frequencies. Similarly, the patient interface module 50 includes one or more connectors 50b. At least one cable 52 connects the patient interface module 50 with console 20, via connectors 20b and 50b.

In this embodiment, the patient interface module 50 includes an isolated localization drive system 54, a set of patch electrodes 56, and one or more reference electrodes 58. The isolated localization drive system 54 isolates localization signals from the rest of system 100 to prevent current leakage (e.g. signal loss) resulting in performance degradation. In some embodiments, the isolation of the localization signals from the remainder of the system comprises a range of impedance greater than 100 kiloohms, such as approximately 500 kiloohms at the localization frequencies. The isolation of the localization drive system 54 can minimize drift in localization positions and maintain a high degree of isolation between axes (as described herebelow). The localization drive system 54 can operate as a current, voltage, magnetic, acoustic, or other type of energy modality drive. The set of patch electrodes 56 and/or one or more reference electrodes 58 can consist of conductive electrodes, magnetic coils, acoustic transducers, and/or other type of transducer or sensor based on the energy modality employed by the localization drive system 54. Additionally, the isolated localization drive system 54 maintains simultaneous output on all axes (e.g. a localization signal is present on each axis electrode pair, while also increasing the effective sampling rate at each electrode position). In some embodiments, the localization sampling rate comprises a rate between 10 kHz and 20 MHz, such as a sampling rate of approximately 625 kdHz.

In some embodiments, the set of patch electrodes 56 include three (3) pairs of patch electrodes: an “X” pair having two patch electrodes placed on opposite sides of the ribs (X1, X2); a “Y” pair having one patch electrode placed on the lower back (Y1) and one patch electrode placed on the upper chest (Y2); and a “Z” pair having one patch electrode placed on the upper back (Z1) and one patch electrode placed on the lower abdomen (Z2). The patch electrode 56 pairs can be placed on any orthogonal and/or non-orthogonal sets of axes. In the embodiment of FIG. 1, the placement of electrodes is shown on patient P, where electrodes on the back are shown in dashed lines.

The reference patch electrode 58 can be placed on the lower back/buttocks. Additionally, or alternatively, a reference catheter can be placed within a body vessel, such as a blood vessel in and/or proximate the lower back/buttocks.

The placement of electrodes 56 defines a coordinate system made up of three axes, one axis per pair of patch electrodes 56. In some embodiments, the axes are non-orthogonal to a natural axis of the body, i.e., non-orthogonal to head-to-toe, chest-to-back, and side-to-side (e.g. rib-to-rib). The electrodes can be placed such that the axes intersect at an origin, such as an origin located in the heart. For instance, the origin of the three intersecting axes can be centered in an atrial volume. System 100 can be configured to provide an “electrical zero” that is positioned outside of the heart, such as by locating a reference electrode 58 such that the resultant electrical zero is outside of the heart (e.g. to avoid crossing from a positive voltage to a negative voltage at one or more locations being localized).

As described above, a patch pair can operate differentially, such as when neither patch 56 in a pair operates as a reference electrode, and are both driven by system 100 to generate the electrical field between the two. Alternatively or additionally, one or more of the patch electrodes 56 can serve as the reference electrode 58, such that they operate in a single ended mode. One of any pair of patch electrodes 56 can serve as the reference electrode 58 for that patch pair, forming a single-ended patch pair. One or more patch pairs can be configured to be independently single-ended. One or more of the patch pairs can share a patch as a single-ended reference or can have the reference patches of more than one patch pair electrically connected.

Through processing performed by console 20, the axes can be transformed (e.g. rotated) from a first orientation (e.g. a non-physiological orientation based on the placement of electrodes 56) to a second orientation. The second orientation can comprise a standard Left-Posterior-Superior (LPS) anatomical orientation, such as when the “x” axis is oriented from right to left of the patient, the “y” axis is oriented from the anterior to posterior of the patient, and the “z” axis is oriented from caudal to cranial of the patient. Placement of patch electrodes 56 and the non-standard axes defined thereby can be selected to provide improved spatial resolution when compared to patch electrode placement resulting in a normal physiological orientation of the resulting axes (e.g. due to preferred tissue characteristics between electrodes 56 in the non-standard orientation). For example, non-standard electrode 56 placement can result in reducing the negative effects of the low-impedance volume of the lungs on the localization field. Furthermore, electrode 56 placement can be selected to create axes which pass through the body of the patient along paths of equivalent, or at least similar, lengths. Axes of similar length will possess more similar energy density per unit distance within the body, yielding a more uniform spatial resolution along such axes. Transforming the non-standard axes into a standard orientation can provide a more straightforward display environment for the user. Once the desired rotation is achieved, each axis can be scaled, such as when made longer or shorter, as needed. The rotation and scaling are performed based on comparing pre-determined (e.g. expected or known) electrode array 12 shape and relative dimensions, with measured values that correspond to the shape and relative dimensions of the electrode array in the patch electrode established coordinate system. For example, rotation and scaling can be performed to transform a relatively inaccurate (e.g. uncalibrated) representation into a more accurate representation. Shaping and scaling the representation of the electrode array 12 can adjust, align, and/or otherwise improve the orientation and relative sizes of the axes for far more accurate localization.

The electrical reference electrode(s) 58 can be or at least include a patch electrode and/or an electrical reference catheter, which can function as a patient “analog ground” reference. A patch electrode 58 can be placed on the skin, and can act as a return for current for defibrillation (e.g. provide a secondary purpose). An electrical reference catheter can include a unipolar reference electrode used to enhance common mode rejection. The unipolar reference electrode, or other electrodes on a reference catheter, can be used to measure, track, correct, and/or calibrate physiological, mechanical, electrical, and/or computational artifacts in a cardiac signal. In some embodiments, these artifacts are due to respiration, cardiac motion, and/or artifacts induced by applied signal processing, such as filters. Another form of an electrical reference catheter can be an internal analog reference electrode, which can act as a low noise “analog ground” for all internal catheter electrodes. Each of these types of reference electrodes can be placed in relatively similar locations, such as near the lower back in an internal blood vessel (as a catheter) and/or on the lower back (as a patch). In some embodiments, system 100 comprises a reference catheter 58 including a fixation mechanism (e.g. a user activated fixation mechanism), which can be constructed and arranged to reduce displacement (e.g. accidental or otherwise unintended movement) of one or more electrodes of the reference catheter 58. The fixation mechanism can comprise a mechanism selected from the group consisting of: spiral expander; spherical expander; circumferential expander; axially actuated expander; rotationally actuated expander; and combinations of two or more of these.

In some embodiments, console 20 includes a defibrillation (DFIB) protection module 22 connected to connector 20a, which is configured to receive cardiac information from the catheter 10. The DFIB protection module 22 is configured to have a precise clamping voltage and a reduced (e.g. minimum) capacitance. Functionally, the DFIB protection module 22 acts a surge protector, configured to protect the circuitry of console 20 during application of high energy to the patient, such as during defibrillation of the patient (e.g. using a standard defibrillation device).

The DFIB protection module 22 can be coupled to three signal paths, a bio-potential (BIO) signal path 30, a localization (LOC) signal path 40, and an ultrasound (US) signal path 60. Generally, the BIO signal path 30 filters noise and preserves the recorded bio-potential data, and also enables the bio-potential signals to be read (e.g. successfully recorded) while ablating (e.g. delivery of RF energy to tissue), which is not the case in other systems. Generally, the LOC signal path 40 allows high voltage inputs, while filtering noise from received localization data. Generally, the US signal path 60 acquires range data from the physical structure of the anatomy using the ultrasound transducers 12b for generation of a 2D or 3D digital model of the heart chamber HC, which can be stored in memory.

The BIO signal path 30 includes an RF filter 31 coupled to the DFIB protection module 22. In this embodiment, the RF filter 31 operates as a low-pass filter having a high input impedance. The high input impedance is preferred in this embodiment because it minimizes the loss of voltage from the source (e.g. catheter 10), thereby better preserving the received signals (e.g. during RF ablation). The RF filter 31 is configured to allow bio-potential signals from the electrodes 12a on catheter 10 to pass through RF filter 31 (e.g. passing frequencies less than 500 Hz), such as frequencies in the range of 0.5 Hz to 500 Hz. However, high frequencies, such as high voltage signals used in RF ablation, are filtered out from the bio-potential signal path 30. RF filter 31 can comprise a corner frequency between 10 kHz and 50 kHz.

A BIO amplifier 32 can comprise a low noise single-ended input amplifier that amplifies the RF filtered signal. A BIO filter 33 (e.g. a low pass filter) filters noise out of the amplified signal. BIO filter 33 can comprise an approximately 31 kHz filter. In some embodiments, BIO filter 33 comprises an approximately 7.5 kHz filter, such as when system 100 is configured to accommodate pacing of the heart (e.g. to avoid significant signal loss and/or degradation during pacing of the heart).

BIO filter 33 can include differential amplifier stages used to remove common mode power line signals from the bio-potential data. This differential amplifier can implement a baseline restore function which removes DC offsets and/or low frequency artifacts from the bio-potential signals. In some embodiments, this baseline restore function comprises a programmable filter which can comprise one or more filter stages. In some embodiments, the filter includes a state dependent filter. Characteristics of the state dependent filter can be based on a threshold and/or other level of a parameter (e.g. voltage), with the filter rate varied based on the filter state. Components of the baseline restore function can incorporate noise reduction techniques such as dithering and/or pulse width modulation of the baseline restore voltage. The baseline restore function can also determine, by measurement, feedback, and/or characterization, the filter response of one or more stages. The baseline restore function can also determine and/or discriminate the portions of the signal representing a physiological signal morphology from an artifact of the filter response and computationally restore the original morphology, or a portion thereof. In some embodiments, the restoration of the original morphology can include subtraction of the filter response directly and/or after additional signal processing of the filter response, such as via static, temporally-dependent, and/or spatially-dependent weighting, multiplication, filtering, inversion, and combinations of these. In some embodiments, the baseline restore function is implemented in BIO filter 33, BIO processor 36, or both.

The LOC signal path 40 includes a high voltage buffer 41 coupled to the DFIB protection module 22. In this embodiment, the high voltage buffer 41 is configured to accommodate the relatively high voltages used in treatment techniques, such as RF ablation voltages. For example, the high voltage buffer can have 100V power-supply rails. In some embodiments, each high voltage buffer 41 has a high input impedance, such as an impedance of 100 kiloohms to 10 megaohms at the localization frequencies. In some embodiments, all high voltage buffers 41, taken together as a total parallel electrical equivalent, also has a high input impedance, such as an impedance of 100 kiloohms to 10 megaohms at the localization frequencies. In some embodiments, the high voltage buffer 41 has a bandwidth that maintains good performance over a range of high frequencies, such as frequencies between 100 kilohertz and 10 megahertz, such as frequencies of approximately 2 megahertz. In some embodiments, the high voltage buffer 41 does not include a passive RF filter input stage, such as when the high voltage buffer 41 has a ±100V power-supply. A high frequency bandpass filter 42 can be coupled to the high voltage buffer 41, and can have a passband frequency range of about 20 kHz to 80 kHz for use in localization. In some embodiments, the filter 42 has low noise with unity gain (e.g. a gain of 1 or about 1).

The US signal path 60 comprises an US isolation multiplexer, MUX 61, a US transformer with a Tx/Rx switch, US transformer 62, a US generation and detection module 63, and an US signal processor 66. The US isolation MUX 61 is connected to the DFIB protection module 22, and is used for turning on/off the US transducers 12b, such as in a predetermined order or pattern. The US isolation MUX 61 can be a set of high input impedance switches that, when open, isolate the US system and remaining US signal path elements, decoupling the impedance to ground (through the transducers and the US signal path 60) from the input of the LOC and BIO paths. The US isolation MUX 61 also multiplexes one transmit/receive circuit to one or more multiple transducers 12b on the catheter 10. The US transformer 62 operates in both directions between the US isolation MUX 61 and the US generation and detection module 63. US transformer 62 isolates the patient from the current generated by the US transmit and receive circuitry in module 63 during ultrasound transmission and receiving by the US transducers 12b. The US transformer 62 can be configured to selectively engage the transmit and/or receive electronics of module 63 based on the mode of operation of the transducers 12b, for example by using a transmit/receive switch. That is, in a transmit mode, the module 63 receives a control signal from a US processor 66 (within a data processor 26) that activates the US signal generation and connects an output of the Tx amplifier to US transformer 62. The US transformer 62 couples the signal to the US isolation MUX 61 which selectively activates the US transducers 12b. In a receive mode, the US isolation MUX 61 receives reflection signals from one or more of the transducers 12b, which are passed to the US transformer 62. The US transformer 62 couples signals into the receive electronics of the US generation and detection module 63, which in-turn transfers reflection data signals to the US processor 66 for processing and use by the user interface 27 and display 27a. In some embodiments, processor 66 commands MUX 61 and US transformer 62 to enable transmission and reception of ultrasound to activate one or more of the associated transducers 12b, such as in a predetermined order or pattern. The US processor 66 can include, as examples, detection of a single, first reflection, the detection and identification of multiple reflections from multiple targets, the determination of velocity information from Doppler methods and/or from subsequent pulses, the determination of tissue density information from the amplitude, frequency, and/or phase characteristics of the reflected signal, and combinations of one or more of these.

An analog-to-digital converter (ADC) 24 is coupled to the BIO filter 33 of the BIO signal path 30 and to the high frequency filter 42 of the LOC signal path 40. Received by the ADC 24 is a set of individual time-varying analog bio-potential voltage signals, one for each electrode 12a. These bio-potential signals have been differentially referenced to a unipolar electrode for enhanced common mode rejection, filtered, and gain-calibrated on an individual channel-by-channel basis, via BIO signal path 30. Received by the ADC is also a set of individual time-varying analog localization voltage signals for each axis of each patch electrode 56, via LOC signal path 40, which are output to the ADC 24 as a collection of 48 (in this embodiment) localization voltages measured at a single time for the electrodes 12a. The ADC 24 has high oversampling to allow noise shaping and filtering, e.g. with an oversampling rate of about 625 kHz. In some embodiments, sampling is performed at or above the Nyquist frequency of system 100. The ADC 24 is a multi-channel circuit that can combine BIO and LOC signals or keep them separate. In one embodiment, as a multi-channel circuit, the ADC 24 can be configured to accommodate 48 localization electrodes 12a and 32 auxiliary electrodes (e.g. for ablation or other processes), for a total of 80 channels. In other embodiments, more or less channels can be provided. In FIG. 1, for example, almost all of the elements of console 20 can be duplicated for each channel (e.g. except for the UI system 27). For example, console 20 can include a separate ADC for each channel, or an 80 channel ADC. In this embodiment, signal information from the BIO signal path 30 and the LOC signal path 40 are input to and output from the various channels of the ADC 24. Outputs from the channels of the ADC 24 are coupled to either the BIO signal processing module 34 or the LOC signal processing module 44, which pre-process their respective signals for subsequent processing as described herebelow. In each case, the preprocessing prepares the received signals for the processing by their respective dedicated processors discussed herebelow. The BIO signal processing module 34 and the LOC signal processing module 44 can be implemented in firmware, in whole or in part, in some embodiments.

The bio-potential signal processing module 34 can provide gain and offset adjustment and/or digital RF filtering having a non-dispersive low pass filter and an intermediate frequency band. The intermediate frequency band can eliminate ablation and localization signals. The bio-potential signal processing module 34 can also include digital bio-potential filtering, which can optimize the output sample rate.

Additionally, the bio-potential signal processing module 34 can also include “pace blanking”, which is the blanking of received information during a timeframe when, for example, a physician is “pacing” the heart. Temporary cardiac pacing can be implemented via the insertion or application of intracardiac, intraesophageal, and/or transcutaneous pacing leads, as examples. The goal in temporary cardiac pacing can be to interactively test and/or improve cardiac rhythm and/or hemodynamics. To accomplish the foregoing, active and passive pacing trigger and input algorithmic trigger determinations can be performed (such as by system 100). The algorithmic trigger determination can use subsets of channels, edge detection and/or pulse width detection to determine if pacing of the patient has occurred. Optionally, pace blanking can be applied by system 100 on all channels or subsets of channels, including channels on which detection did not occur.

Additionally, the bio-potential signal processing module 34 can also include specialized filters that remove ultrasound signals and/or other unwanted signals (e.g. artifacts from the bio-potential data). In some embodiments, to perform this filtering, edge detection, threshold detection and/or timing correlations are used.

The localization signal processing module 44 can provide individual channel/frequency gain calibration, IQ demodulation with tuned demodulation phase, synchronous and continuous demodulation (without MUXing), narrow band R filtering, and/or time filtering (e.g. interleaving, blanking, etc.), as discussed herebelow. The localization signal processing module 44 can also include digital localization filtering, which optimizes the output sample rate and/or frequency response.

In this embodiment, the algorithmic computations for the BIO signal path 30, LOC signal path 40, and US signal path 60 are performed in console 20. These algorithmic computations can include but are not limited to: processing multiple channels at one time, measuring propagation delays between channels, turning x, y, z data into a spatial distribution of electrode locations, including computing and applying corrections to the collection of positions, combining individual ultrasound distances with electrode locations to calculate detected endocardial surface points, and constructing a surface mesh from the surface points. The number of channels processed by console 20 can be between 1 and 500, such as between 24 and 256, such as 48, 80, or 96 channels.

A data processor 26, which can include one or more of a plurality of types of processing circuits (e.g. a microprocessor) and memory circuitry, executes computer instructions necessary to perform the processing of the pre-processed signals from the BIO signal processing module 34, localization signal processing module 44, and US TX/RX MUX 61. The data processor 26 can be configured to perform calculations, as well as perform data storage and retrieval, necessary to perform the functions of system 100.

In this embodiment, data processor 26 can include a bio-potential (BIO) processor 36, a localization (LOC) processor 46, and an ultrasound (US) processor 66. The bio-potential processor 36 can perform processing of recorded, measured, or sensed bio-potentials (e.g., from electrodes 12a). The LOC processor 46 can perform processing of localization signals. The US processor 66 can perform image processing of the reflected US signals, (e.g. from transducers 12b).

Bio-potential processor 36 can be configured to perform various calculations. For example, BIO processor 36 can include an enhanced common mode rejection filter, which can be bidirectional to minimize distortion and which can be seeded with a common mode signal. BIO processor 36 can also include an optimized ultrasound rejection filter and be configured for selectable bandwidth filtering. Processing steps for data in US signal path 60 can be performed by bio signal processor 34 and/or bio processor 36.

Localization processor 46 can be configured to perform various calculations. As discussed in more detail herebelow, LOC processor 46 can electronically make (calculate) corrections to an axis based on the known shape of electrode array 12, make corrections to the scaling or skew of one or more axes based on the known shape of the electrode array 12, and perform “fitting” to align measured electrode positions with known possible configurations, which can be optimized with one or more constraints (e.g. physical constraints, such as distance between two electrodes 12a on a single spline, distance between two electrodes 12a on two different splines, maximum distance between two electrodes 12a, minimum distance between two electrodes 12a, and/or minimum and/or maximum curvature of a spline, and the like).

US processor 66 can be configured to perform various calculations associated with generation of the US signal via the US transducers 12b and processing US signal reflections received by the US transducers 12b. US processor 66 can be configured to interact with the US signal path 60 to selectively transmit and receive US signals to and from the US transducers 12b. The US transducers 12b can each be put in a transmit mode and/or a receive mode under control of the US processor 66. The US processor 66 can be configured to construct a 2D and/or 3D image of the heart chamber (HC) within which the electrode array 12 is disposed, using reflected US signals received from the US transducers 12b via the US path 60.

Console 20 can also include localization driving circuitry, including a localization signal generator 28 and a localization drive current monitor circuit 29. The localization drive circuitry provides high frequency localization drive signals (e.g. 10 kHz-1 MHz, such as 10 kHz-100 kHz). Localization using drive signals at these high frequencies reduces the cellular response effect on the localization data (e.g. from blood cell deformation), and/or allows higher drive currents (e.g. to achieve a better signal-to-noise ratio). Signal generator 28 produces a high resolution digital synthesis of a drive signal, (e.g. a sine wave), with ultra-low phase noise timing. The drive current monitoring circuitry provides a high voltage, wide bandwidth current source, which is monitored to measure impedance of the patient P.

Console 20 can also include at least one data storage device 25, for storing various types of recorded, measured, sensed, and/or calculated information and data, as well as program code embodying functionality available from the console 20.

Console 20 can also include a user interface (UI) system 27 configured to output results of the localization, bio-potential, and US processing. UI system 27 can include at least one display 27a to graphically render such results in 2D, 3D, or a combination thereof. In some embodiments, the display 27a includes two simultaneous views of the 3D results with independently configurable view/camera properties, such as view directions, zoom level, pan position, and object properties, such as color, transparency, brightness, luminance, etc. UI System 27 can include one or more user input components, such as a touch screen, a keyboard, a joystick, and/or a mouse.

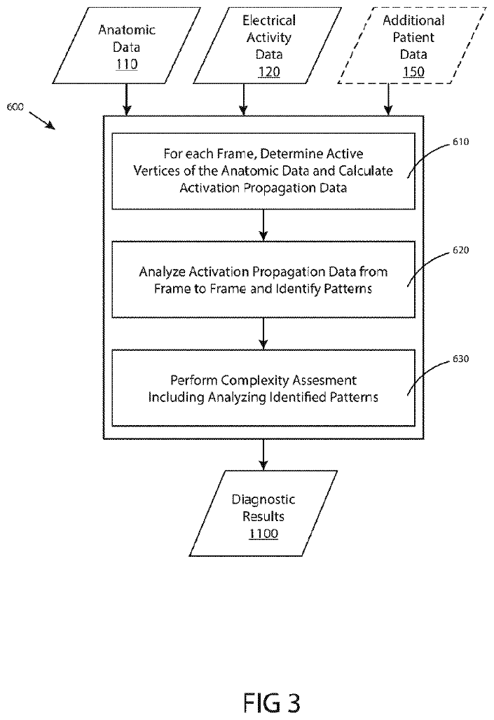

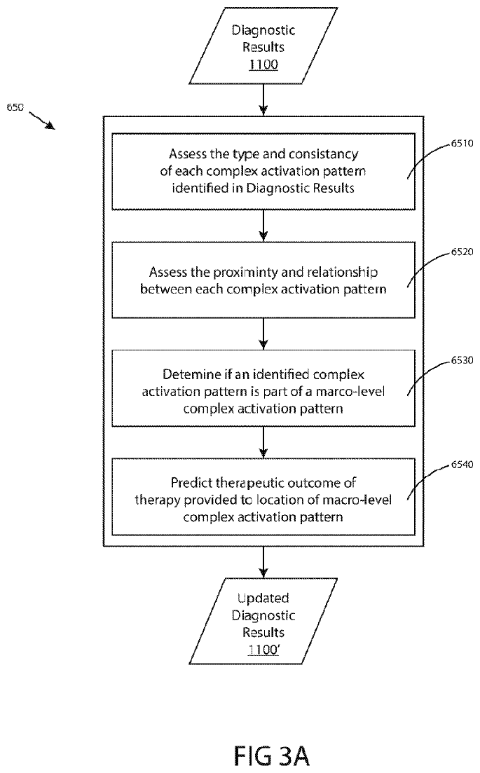

Console 20, or another component of system 100, can include one or more algorithms, such as complexity algorithm 600 shown. Complexity algorithm 600 can comprise an algorithm as described herebelow in reference to FIG. 3. Complexity algorithm 600 can include one or more algorithms, such as one or more of: CV algorithm 200, LRA algorithm 300, LIA algorithm 400, FA algorithm 500, and/or complexity algorithm 600 described herebelow. Complexity algorithm 600 can identify, quantify, categorize, and/or otherwise assess cardiac conduction patterns or characteristics, such as to produce diagnostic information, diagnostic results 1100 herein. Complexity algorithm 600 can produce an assessment, over time and/or space, of complexity and/or an assessment of a variation of complexity over time. In some embodiments, complexity algorithm 600, and/or another algorithm of system 100, comprises a bias. In some embodiments, the algorithm comprises a bias toward false positives (e.g. a bias towards falsely identifying a non-complex region as being complex, versus not classifying a complex region as being complex). In some embodiments, the algorithm comprises a bias toward false negatives. In some embodiments, an algorithm of system 100 comprises a bias that is set and/or adjusted (“set” herein) by a clinician, such as to bias system 100 toward a particular preference of the clinician.

Complexity, as determined by the algorithms of the present inventive concepts, includes any deviation from the expected or normal behavior of what would otherwise be a simple, repetitive, and consistent pattern of electrical activity. In cardiac electrical activity, the expected or normal behavior of the heart chamber is consistent, repetitive, and coordinated activation of the tissue, called sinus rhythm, that initiates at a location (e.g. the sino-atrial node) and propagates along the chamber smoothly. Complexity includes any deviation that disrupts the consistency (e.g. time, amplitude, direction, and/or repetition rate of activation), and/or coordination/order (e.g. time and/or direction of activation). Regions of tissue may self-initiate electrical activation (automaticity), interrupting otherwise coordinated activation. Regions of tissue that may be compromised, scarred, diseased and/or possess otherwise heterogenous characteristics (e.g. fibrosis, varying fiber orientations, varying endocardial to epicardial pathways, and the like) can create complexity of cardiac activity, as described hereabove. A region that creates complexity may disrupt the expected conduction in a consistent way. For example, conduction may be redirected in a different direction and with a reduction in amplitude, but can do so in the same way for each activation. Alternatively, a region that exhibits complexity (e.g. as identified by an algorithm of system 100), may disrupt the expected conduction in a stochastic or probabilistic way (e.g. seemingly random variation), but in a way that possesses a recognizable statistical behavior in how it disrupts conduction. For example, modified conduction can be identified through a region in one characteristic manner for X % of the time, and in a second, different characteristic manner, for Y % of the time. In some embodiments, for Z % (where Z<100) of the time, the activation exhibits normal conduction, however the region is still identified by system 100 as complex due to modified conduction, in one or more forms, for some portion of the time.

The algorithms of the present inventive concepts can be configured to identify when multiple regions of complexity interact, or otherwise couple, in ways that create further complexity across the cardiac chamber, thereby compounding the degree of global complexity over the heart chamber, such as is described herebelow in reference to FIG. 3A. Because the cardiac tissue has propagative properties with a refractory (non-active) period, complexity that impacts the order and timing of activation can have lasting/persisting effects on later activations in time, and across a broad spatial area. Therefore, as the number of unique or discrete zones of automaticity or heterogeneity increases (tissue-mediated complexity), the resulting electrical activation becomes increasingly complex (e.g. a compounding of both tissue-mediated complexity and coupling-related complexity), tied together in time and space by the propagating nature of cardiac tissue, established by the variations in conduction preceding, and affecting variations in conduction to follow. As the complexity increases, the ability to identify the tissue-mediated complexity from the coupling-related complexity based on simple electrical measurements becomes more difficult. System 100 can be configured to gather more information over time and across space (e.g. simultaneously), with the additional information gathered to aid in one or more algorithms decoding the complexity locally, regionally, and globally across the chamber.

Complexity algorithm 600 can perform a complexity assessment based on calculated electrical activity data 120b that represents multiple vertices, such as when the associated recorded electrical activity data 120a comprises data recorded from at least three recording locations within a heart chamber (e.g. on and/or offset from the heart wall). In some embodiments, the recorded electrical activity data 120a includes at least one location offset from the walls of the heart (e.g. at least one non-contact recording). In some embodiments, the recorded electrical activity data 120a includes at least one location on a wall of the heart (e.g. at least one contact recording). In some embodiments, the recorded electrical activity data 120a includes at least one location offset from the walls of the heart, and at least one location on a wall of the heart (e.g. at least one contact and one non-contact recording, a ‘hybrid’). In some embodiments, for each location on the heart wall in which a contact-based measurement is made, system 100 is biased to categorize that location as a vertex.

In some embodiments, algorithm 600 comprises a second algorithm configured to calculate surface charge data and/or dipole density data for each of the multiple vertices, based on the recorded electrical activity data 120a (e.g. recorded voltages), such as when the complexity analysis is based on surface charge data and/or dipole density data. Surface charge data and/or dipole density data can be calculated as described in applicant's U.S. Pat. No. 8,417,313, titled “METHOD AND DEVICE FOR DETERMINING AND PRESENTING SURFACE CHARGE AND DIPOLE DENSITIES ON CARDIAC WALLS”, issued Apr. 9, 2013, and U.S. Pat. No. 8,512,255, titled “DEVICE AND METHOD FOR THE GEOMETRIC DETERMINATION OF ELECTRICAL DIPOLE DENSITIES ON THE CARDIAC WALL”, issued Aug. 20, 2013, the content of each of which is incorporated herein by reference in its entirety for all purposes. In some embodiments, algorithm 600 comprises a third algorithm that converts the surface charge data and/or the dipole density data into surface voltage data, such as when the complexity analysis is based on the surface voltage data.

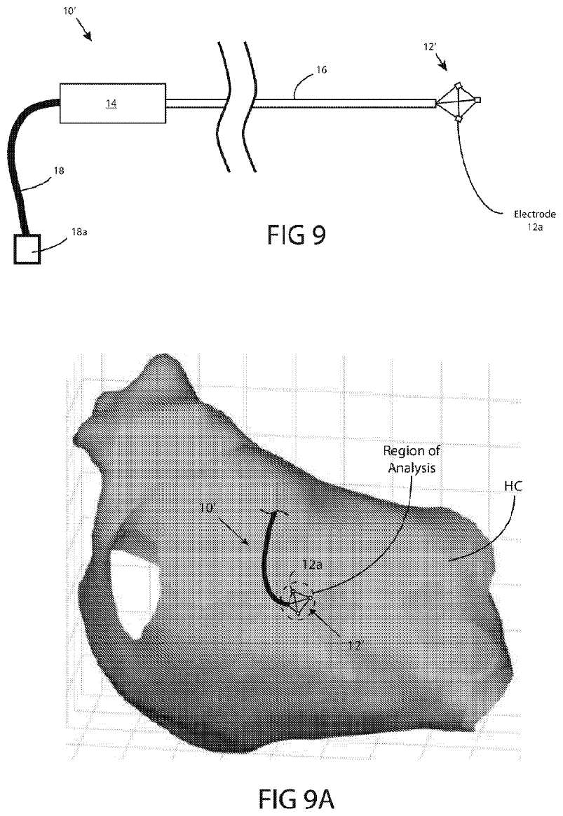

In some embodiments, algorithm 600 performs a complexity assessment over a relatively small portion of the patient's heart (e.g. a relatively small portion of a patient's heart chamber), such as a portion that represents no more than 7 cm² of the heart wall, such as no more than 4 cm², such as no more than 1 cm². In these embodiments, electrical activity can be recorded (e.g. by electrodes 12a) from at least three recording locations, and calculated electrical activity data 120b can be determined for at least 3 vertices (as described herein). In some embodiments, the at least three recording locations comprise at least three locations on the heart wall (e.g. via a contact-based recording). In some embodiments, at least one recording location is offset from the heart wall (e.g. non-contact mapping). In some embodiments, algorithm 600 performs the small portion complexity assessment using voltage data and/or dipole density data. In some embodiments, analysis of a small portion of the patient's heart is performed with system 100 and the associated method described herebelow in reference to FIGS. 9 and 9A.

In some embodiments, algorithm 600 performs a complexity assessment over a moderate or large portion of the patient's heart, such as a portion of the patient's heart representing at least 7 cm² of heart wall tissue (e.g. wall tissue of an atria of the heart), such as a minimum surface area of 1 cm², such as 4 cm², such as 7 cm². In these embodiments, electrical activity can be recorded (e.g. by electrodes 12a) from at least 24 locations within the heart (e.g. within a single heart chamber), and calculated electrical activity data 120b can be determined for at least 64 vertices. In some embodiments, electrical activity can be recorded from at least 24 heart wall locations (e.g. via a contact-based recording), with or without additional recordings made offset from the heart wall (e.g. in the flowing blood via a non-contact-based recording). In these embodiments, electrical activity can be recorded from at least 48 heart wall locations, or at least 64 heart locations. In some embodiments, electrical activity is recorded from both locations on the heart wall and offset from the heart wall, such as when data is recorded from at least 24, at least 48, or at least 54 contact and non-contact locations within the heart chamber. In these embodiments, calculated electrical activity data 120b can be determined for at least 100 vertices, such as at least 500, at least 3000, and/or at least 5000 vertices.

In some embodiments, the complexity algorithm 600 incorporates data through various depths (e.g. layers) of tissue. In thicker tissues, electrical conduction can vary through the thickness. The stretch and/or strain of the tissue can also have an impact on the conduction properties of the tissue. Measuring, recording, and/or calculating electrical data or biomechanical data through the depth of tissue can be used to improve the accuracy and/or specificity of complexity algorithm 600. In some embodiments, surface charge density and/or dipole density is calculated through a thickness of tissue of the cardiac chamber, with the calculated data used as input to complexity algorithm 600. In some embodiments, surface charge density and/or dipole density are determined as described in applicant's co-pending U.S. patent application Ser. No. 15/926,187, titled “DEVICE AND METHOD FOR THE GEOMETRIC DETERMINATION OF ELECTRICAL DIPOLE DENSITIES ON THE CARDIAC WALL”, filed Mar. 20, 2018, the content of which is incorporated herein by reference in its entirety for all purposes.

Complexity algorithm 600 can assess the variation of one or more characteristics, such as electrical, mechanical, functional, and/or physiologic characteristics of the heart that vary in time, space, magnitude and/or state. Studies of cardiac behavior, function, and other characteristics, over the last several decades have yielded a substantive understanding of what is considered “normal”. Cardiac conditions such as cardiac arrhythmias exhibit variations from the norm in many ways, and these variations can be quantified, qualified, and/or otherwise assessed by complexity algorithm 600.

In some embodiments, variations in time or temporal repetition and/or stability (e.g. measures of temporal regularity and/or irregularity) indicate the presence of a cardiac arrhythmia. Electrical characteristics (e.g. cycle length, dominant frequency, harmonic organization, fractionation or measures of waveform “energy”, Shannon entropy, waveform deflections within a time window, temporal wave recurrence, regularity, and/or higher order statistics of the electrical data, such as kurtosis) can be measured or otherwise determined by system 100, and these characteristics can be included in the assessment performed by complexity algorithm 600. System 100 can determine these variables using tools such as: interval analysis; Fourier, Hilbert or other transforms; wavelet analysis; and combinations of these.

Mechanical and/or functional (“mechanical” herein) characteristics assessed by algorithm 600 can include deflection timing of the heart wall over time. In some embodiments, system 100 determines, and algorithm 600 assesses a combination of electrical, and/or mechanical data, such as electro-mechanical delay (e.g. which can also vary as a function of time).

In some embodiments, algorithm 600 assesses a variation in magnitude and/or state of a characteristic determined by system 100. For example, electrical characteristics assessed can include an assessment of electrical activity at a cardiac surface, such as an assessment of: rms amplitude; peak-to-peak amplitude; peak-negative amplitude; and combinations of these. Mechanical characteristics assessed can include total or average deflection of the heart wall through one or more phases of the cardiac cycle. In some embodiments, a combination of electrical and mechanical data includes ratios of electrical magnitude to mechanical magnitude and/or functional efficiency.

In some embodiments, algorithm 600 assesses a variation over space or in direction of one or more characteristics. For example, electrical characteristics assessed can include: directional bipoles formed in different directions (e.g. determined from data recorded by unipolar electrodes); conduction velocity direction; spatial wave analysis; and combinations of these. In some embodiments, a Laplacian operator can be applied to electrical activity data 120a recorded from a multi-polar and/or omni-polar catheter to provide calculated data for algorithm 600 to assess.

In some embodiments, algorithm 600 assesses variations in one or more characteristics, in two or more of: time; space; magnitude; and/or state. In some embodiments, algorithm 600 assesses two or more of these that vary simultaneously, such as a temporospatial variation. In these embodiments, algorithm 600 can assess electrical characteristics to determine if a pattern of interest occurs (e.g. focal, rotational, irregular, directional, and/or timing patterns). Algorithm 600 can assess temporospatial features or patterns, such as an activation sequence or conduction pattern that exhibits one or more of the following characteristics: propagation that ‘breaks out’ through a confined ‘gap’ or opening, regionally constrained pivoting re-entry, and other irregular conduction patterns (e.g. patterns that vary in time and space), rotation about a central core or obstacle, and/or focal activation spreading from a single location. Algorithm 600 can include an assessment of changes in conduction velocity (e.g. magnitude and/or direction). Algorithm 600 can perform any qualitative and/or quantitative analysis of one or more of these characteristics, such as to provide an assessment of complexity.

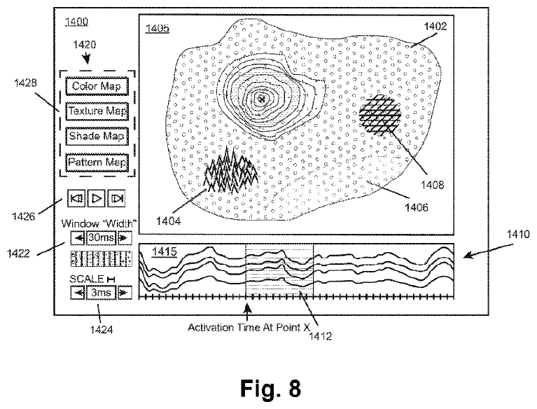

The complexity assessment provided by algorithm 600 can comprise a binary measure of whether the complexity occurred at one or more times at each location (e.g. each vertex) assessed. The complexity assessment provided by algorithm 600 can comprise a static level of complexity across a time period (e.g. a sum, average, median, variance, standard deviation, and/or percentile level). Static levels determined can be thresholded to calculate and/or display a subset range of the static data. The complexity assessment provided by algorithm 600 can comprise an assessment of change in complexity over time (e.g. over one or more time periods), such as an assessment of changes in rate, frequency, degree, percentile and/or probability. Complexity algorithm 600 can perform multiple complexity assessments in sequence, such as using a “rolling window” as described herebelow in reference to FIG. 8. The multiple complexity assessments can include an assessment of a static quantity of complexity over time.

Complexity algorithm 600 can assess complexity (e.g. changes in complexity) and produce results (e.g. diagnostic results 1100) that are used for multiple purposes. For example, algorithm 600 can provide an assessment of the stability and/or consistency of complexity, and/or other arrhythmogenic conditions, based on an analyzed recording duration of a few minutes or less (e.g. a duration of less than 10 minutes). The assessment can differentiate areas of consistent complexity versus transient or intermittent complexity. Regions of consistency can be correlated to specific tissue substrate characteristics. In the cardiac system, areas where the tissue substrate is anisotropic, heterogeneous, abnormal or diseased may consistently create variation and/or complexity in the electrical activity at that tissue location. However, areas of normal tissue may also see variation or other complexity (wave collisions, interference, fusion, functional block, and the like) resulting from downstream interaction of complex propagating wavefronts created by anisotropic areas of the tissue substrate. This complexity is a “functional” effect where the electrophysiological interactions of propagating waves can cause these waves to interfere or interact with one another in complex ways, often intermittently. Because cardiac tissue remains in a refractory (unable to be re-activated) state for a period of time following each activation, the functional effect occurs not only at the moment when a wave of activation passes, but for an extended period after it has passed. The net result is that complexity of cardiac tissue activation, as identified by complexity algorithm 600, can also occur in areas where the tissue itself is not abnormal or diseased, but is rather due to the prior complex interactions that occurred at other tissue locations. Fixed, substrate-mediated complexity (or mechanisms) will probabilistically re-occur at the same location. Functional complexity may vary in location and frequency of occurrence at a given location. Complexity algorithm 600 can be configured to assess the consistency, stability, repeatability, and/or pattern of complexity to differentiate between fixed, substrate-mediated complexity vs. functional complexity, as described herebelow in reference to FIG. 3A.

Complexity algorithm 600 can be used to determine electrical changes resulting from a delivered therapy (e.g. an RF or other cardiac ablation, such as a therapy provided by treatment subsystem 800, as described herebelow). Comparison of complexity and/or consistency of complexity (“complexity” herein) before and after a therapeutic activity or interval can be used to indicate the electrophysiological impact of the delivered therapy. Algorithm 600 can provide a comparison in the form of a difference plot. Therapeutic events may be as short as a few seconds (at a single or small number of locations) or up to many minutes (for more extensive maneuvers such as ablative lines, loops, cores, boxes, and the like). The longer the therapeutic activity or interval, the more change may exist in the comparison. In some embodiments, system 100 provides a real time (e.g. during therapy) feedback-loop of cause (therapy) and effect (complexity assessment, such as a change in complexity prior to and after therapy). System 100 can be configured to provide a complexity assessment (e.g. recorded electrical activity data 120a and calculate complexity via algorithm 600) in a relatively short period of time (e.g. less than 10 minutes, or less than 5 minutes), such that the clinician is more likely to reduce therapeutic interval times to assess complexity after each interval. In these embodiments, unnecessary ablations can be avoided and/or overall procedure time can be reduced.

Complexity algorithm 600 can be configured to produce complexity data (e.g. the output of a complexity assessment) in real time, such that the complexity data (e.g. diagnostic results 1100) can be shown dynamically, also in real time. For example, system 100 can record and process electrical activity data 120a, and algorithm 600 can analyze the recorded activity, such as using a rolling window (e.g. as described herebelow in reference to FIG. 8), such as a time window with a duration of between 5 seconds and 60 seconds. Algorithm 600 provides multiple complexity assessments by continuously analyzing recorded electrical activity data 120a over the total duration assessed, with newer data added and oldest data excluded as the electrical activity data 120a recording continues. Complexity assessments (e.g. multiple complexity assessments provided in a video format) can be provided in real time (e.g. with a short processing delay), such as during a treatment (e.g. ablation) to dynamically determine when the treatment has achieved a desired result (e.g. sufficient energy has been delivered to cause the desired effect, such as electrical block), and/or how to modify the therapy to achieve a therapeutic goal or otherwise improve efficiency. Alternatively or additionally, the provided complexity assessments can be visualized (e.g. in a playback mode) one or more times after the associated recording of electrical activity data 120a has ceased, such as to perform additional therapy and/or modify the therapy.

Complexity algorithm 600 can provide complexity assessments based on electrical activity data 120 (and/or additional patient data 150 as described herebelow) recorded during two separate clinical procedures (e.g. a first clinical procedure and a subsequent, second clinical procedure). Algorithm 600 can provide one or more complexity assessments for each clinical procedure, such as to allow a comparison to be made between assessments from two different procedures (e.g. an assessment made by algorithm 600). The second clinical procedure can be separated from the first clinical procedure by days, weeks, months, or years. A comparative assessment made by algorithm 600 can assess the therapeutic effects of the first procedure and the recovery (e.g. healing) of the cardiac tissue or the adaptation of the cardiac tissue in the interim between procedures. Cardiac tissue may adapt in response to the altered electrical characteristics (e.g. altered patterns, rhythms, and the like, such as from electrical remodeling), and/or the altered mechanical characteristics (e.g. function) of the tissue, each as caused by the preceding therapeutic procedure. Techniques used in the second clinical procedure can be based on these above assessments provided by algorithm 600 (e.g. in the form of diagnostic results 1100), such as the tissue response (e.g. the electrical and mechanical response described hereabove) to the therapy provided in the first procedure.

While algorithm 600 has been described hereabove as analyzing electrical activity data 120, in some embodiments, algorithm 600 further includes in its assessment, an analysis of “additional patient data” recorded by system 100 (e.g. the complexity assessment is based on additional patient data 150 recorded by system 100 as well as electrical activity data 120 and anatomical data 110 described hereabove). For example, system 100 can comprise one or more functional elements configured as sensors, such as functional element 99 of catheter 10, functional element 899 of treatment catheter 800 described herebelow, and/or functional element 199 of system 100. Functional element 99 of catheter 10 can comprise one or more sensors positioned on an expandable spline of electrode array 12 (as shown), and/or on shaft 16. Functional element 199 of system 100 can comprise a sensor positioned proximate the patient (e.g. on the skin of the patient or relatively near the patient) and/or a sensor positioned within the patient (e.g. temporarily or chronically positioned under the patient's skin). In some embodiments, one or more electrodes 12a and/or ultrasound transducers 12b are configured to record the additional patient data 150.

In some embodiments, sensor-based functional elements 99, 199, and/or 899 comprises a sensor selected from the group consisting of: an electrode or other sensor for recording electrical activity; a force sensor; a pressure sensor; a magnetic sensor; a motion sensor; a velocity sensor; an accelerometer; a strain gauge; a physiologic sensor; a glucose sensor; a pH sensor; a blood sensor; a blood gas sensor; a blood pressure sensor; a flow sensor; an optical sensor; a spectrometer; an interferometer; a measuring sensor, such as to measure size, distance, and/or thickness; a tissue assessment sensor; and combinations of one, two, or more of these.

Additional patient data recorded by system 100 (e.g. via catheter 10, functional element 199, functional element 899, and/or other sensor of system 100), can include patient mechanical information; patient physiologic information; and/or patient functional information. Additional data recorded by system 100 can include data related to a patient parameter selected from the group consisting of: heart wall motion; heart wall velocity; heart tissue strain; magnitude and/or direction of heart blood flow; vorticity of blood; heart valve mechanics; blood pressure; tissue properties, such as density, tissue characteristics and/or biomarkers for tissue characteristics, such as metabolic activity or pharmaceutical uptake; tissue composition (e.g. collagen, myocardium, fat, connective tissue); and combinations of one, two, or more of these.

As described hereabove, one or more complexity assessments performed by algorithm 600 can be based on this additional patient data, such as when both electrical activity data 120 and additional patient data 150 is included in the analysis performed. In some embodiments, the complexity assessment performed by algorithm 600 comprises an assessment of one or more of: electrical-mechanical delay of tissue; magnitude ratio of an electrical to a mechanical characteristic; and combinations of these.

Additional patient data 150 can also comprise prior data (e.g. data collected during a prior procedure) from the same patient or prior data from a set of historical patients other than the patient being diagnosed or treated. The data can be used to form a computational model into which the existing patient's data is fitted, classified, ranked, prioritized, optimized, and/or otherwise assessed as described above.

Diagnostic results 1100 can comprise measured data and/or data resulting from an analysis of measured data (e.g. an analysis of recorded electrical activity data 120a and/or anatomical data 110). Diagnostic results 1100 can be provided (e.g. provided to a clinician of the patient), in one or more forms, such as when displayed on display 27a, provided audibly (e.g. by a speaker of system 100), and/or provided in a printed report (e.g. by a printer of system 100). Diagnostic results 1100 can be used by a clinician to customize a therapy for the patient, such as to determine at which locations to ablate tissue in a cardiac ablation procedure, such as is described in applicant's co-pending U.S. patent application Ser. No. 14/422,941, titled “CATHETER, SYSTEM AND METHODS OF MEDICAL USES OF SAME, INCLUDING DIAGNOSTIC AND TREATMENT USES FOR THE HEART”, filed Feb. 20, 2015, the content of which is incorporated herein by reference in its entirety for all purposes.

In some embodiments, diagnostic results 1100 are based on a complexity assessment performed by complexity algorithm 600 for a single heart wall location or multiple heart wall locations. The single and/or multiple location diagnostic results 1100 can be presented to a user (e.g. the patient's clinician) in reference to an image of the patient's anatomy (e.g. via display 27a). Diagnostic results 1100 can comprise an assessment of complexity over time, such as an assessment of complexity over a pre-determined time duration.

As described hereabove, system 100 can be configured to perform a medical procedure (e.g. a diagnostic, prognostic, and/or therapeutic procedure) related to an arrhythmia or other cardiac condition of the patient. System 100 can be configured to perform a medical procedure on a patient with a cardiac condition selected from the group consisting of: atrial fibrillation; atrial flutter; atrial tachycardia; atrial bradycardia, ventricular tachycardia; ventricular bradycardia; ectopy; congestive heart failure; angina; arterial stenosis; and combinations of one, two, or more of these. In some embodiments, system 100 performs a medical procedure on a patient that exhibits heterogeneous activation, conduction, depolarization, and/or repolarization that varies in time, space, magnitude, and/or state (e.g. combinations, such as velocity). Electrical activity of the patient's heart may contain patterns that can be detected or mapped by system 100, such as patterns selected from the group consisting of: focal; re-entrant; rotational; pivoting; irregular (e.g. in direction and/or velocity); functional block; permanent block; and combinations thereof.

System 100 can include devices or agents (e.g. pharmaceutical agents), treatment subsystem 800, for treating a patient (e.g. treating one or more cardiac conditions of the patient). In the embodiment shown in FIG. 1, treatment subsystem 800 includes a treatment catheter 850, including shaft 860, which can be configured to be advanced through the patient's vasculature into one or more chambers of the patient heart, using standard interventional techniques. In some embodiments, the distal portion of shaft 860 is advanced into the patient's left atrium via a transseptal sheath, not shown but such as a standard device used in left atrial ablation procedures. Treatment catheter 850 comprises treatment element 870 on the distal end (as shown) or at least the distal portion of shaft 860. Treatment element 870 can comprise one or more treatment elements, such as one or more energy delivery elements configured to deliver energy to ablate cardiac tissue (e.g. ablation energy delivered to the heart wall). Treatment element 870 can include an array (e.g. a linear or other array) of treatment elements. Treatment element 870 can comprise one or more electrodes configured to deliver radiofrequency (RF) or other electromagnetic energy to tissue. In some embodiments, treatment element 870 comprises one or more energy delivery elements configured to deliver energy in a form selected from the group consisting of: electromagnetic energy such as RF energy and/or microwave energy; thermal energy such as heat energy and/or cryogenic energy; light energy such as laser light energy; sound energy such as ultrasound energy; chemical energy; mechanical energy; and combinations of these. In some embodiments, treatment element 870 comprises one or more agent delivery elements (e.g. one or more needles, iontophoretic elements, and/or fluid jets) configured to deliver an agent (e.g. a pharmaceutical agent) into cardiac tissue or other tissue of the patient.

Treatment subsystem 800 can further include an energy delivery unit, EDU 810 which provides energy to the one or more treatment elements 870. EDU 810 can provide one or more forms of energy selected from the group consisting of: electromagnetic energy such as RF energy and/or microwave energy; thermal energy such as heat energy and/or cryogenic energy; light energy such as laser light energy; sound energy such as ultrasound energy; chemical energy; mechanical energy; and combinations of these. Alternatively or additionally, EDU 810 can provide an agent to one or more treatment elements 870, such as when treatment elements 870 comprise an agent delivery element as described hereabove.

In some embodiments, treatment subsystem 800, treatment catheter 850, and/or EDU 810 are of similar construction and arrangement to the similar components described in applicant's co-pending U.S. patent application Ser. No. 14/422,941, titled “CATHETER, SYSTEM AND METHODS OF MEDICAL USES OF SAME, INCLUDING DIAGNOSTIC AND TREATMENT USES FOR THE HEART”, filed Feb. 20, 2015, the content of which is incorporated herein by reference in its entirety.

In some embodiments, treatment subsystem 800 is used to treat the patient based on the diagnostic results 1100 (e.g. results which are based on complexity assessment provided by algorithm 600). For example, ablation energy can be delivered to the heart wall at one or more locations (e.g. one or more vertices described hereabove), where the complexity assessment determines if a complexity level for a location exceeds (e.g. is above) a threshold, and therapy is delivered to all locations where the threshold is exceeded. In some embodiments, one vertex is selected for ablation, in a region of multiple vertices, where system 100 (e.g. via algorithm 600) determines a maximum complexity level to exist (e.g. a “local maximum” is ablated), and where the maximum complexity level can be an absolute maximum or a relative maximum.

In some embodiments, therapy provided by system 100 (e.g. ablation energy delivered to one or more vertices) is delivered in a closed-loop fashion, such as in a manual (clinician driven), automated (e.g. system 100 driven), and/or semi-automated (e.g. combined clinician and system 100 driven) mode. Closed-loop operation can include: manipulation of treatment element 870 to a location to be treated (e.g. via clinician manipulated and/or system 100 robotically manipulated treatment device 850); and/or setting of energy level to be delivered.

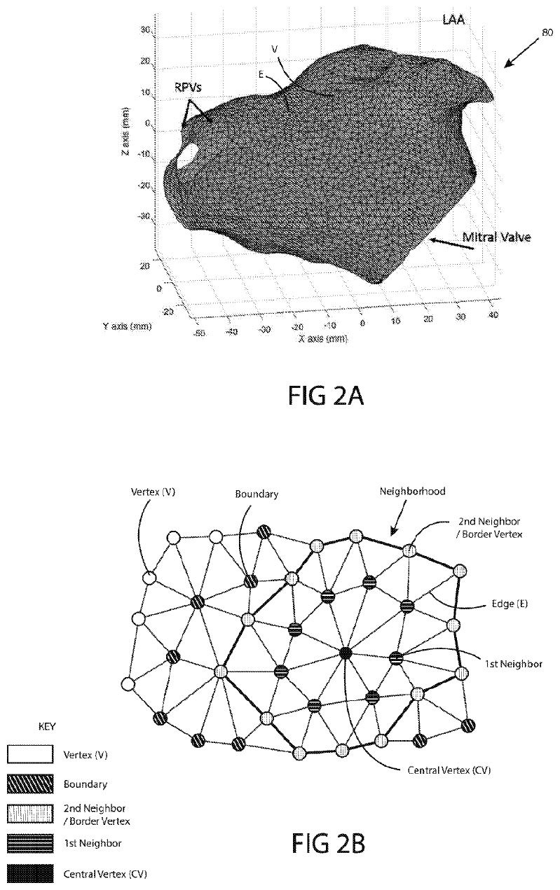

Referring now to FIGS. 2A and 2B, a visual representation of a data structure and a portion of the data structure are illustrated, respectively, consistent with the present inventive concepts. System 100, as describe hereabove, can measure and record the size and shape of a heart chamber HC, for example to provide an approximation of the shape of chamber HC at diastole. In some embodiments, system 100 measures chamber HC via ultrasound transducers 12b of catheter 10, and the measurement information can then be processed by processor 26, and recorded as a set of information defined by a data structure as described herebelow. Alternatively or additionally, system 100 can include other imaging elements and/or devices to provide cardiac anatomy information to processor 26. The processed information provided by processor 26 (e.g. anatomic data 110) can be stored as a set of nodes, each node comprising a vertex V of a geometric representation of the anatomy, for example a triangular mesh representing the chamber HC, mesh 80 shown. Each vertex V in mesh 80 is connected to its neighboring vertices V by edges E, edges of the polygons (e.g. triangles) that define mesh 80.

Any vertex V can be defined as a central vertex CV. For a central vertex CV, a “neighborhood” of surrounding vertices V can be defined (“neighborhood” or “neighborhood of vertices” herein). For example, a neighborhood of first neighbors can comprise central vertex CV as well as all vertices V connected by a single edge E to central vertex CV. Furthermore, a neighborhood of second neighbors can further comprise all vertices V connected by a single edge E to any of the first neighbors of central vertex CV. A two-edge-connected neighborhood is illustrated in FIG. 2B. A multiple-edge-connected neighborhood can be defined by the number of edges from central vertex CV (e.g. in a five-edge-connected neighborhood, each included vertex V is within five edges of central vertex CV). As used herein, a “border vertex” can be defined as a vertex V included within the neighborhood, that is located at a particular number of edges from the central vertex (i.e. the number of edges that defines the size of the neighborhood). A “boundary vertex” can be defined as a vertex V one-edge-connected to a border vertex, but not included within the neighborhood (a vertex that is within one edge-connection of a border vertex but not within the neighborhood).

For each vertex V, information corresponding to its anatomic location can be recorded and stored by system 100. For example, for an instance in time, bio-potential data measured by system 100 can be processed and recorded as a set of values, each corresponding to a vertex V, for that instance in time, (a “frame” of data). System 100 can be configured to record bio-potential or other data for an extended period (e.g. 100 ms to 500 ms), represented by multiple sequential frames, each containing time related information correlating to the vertices V of mesh 80.

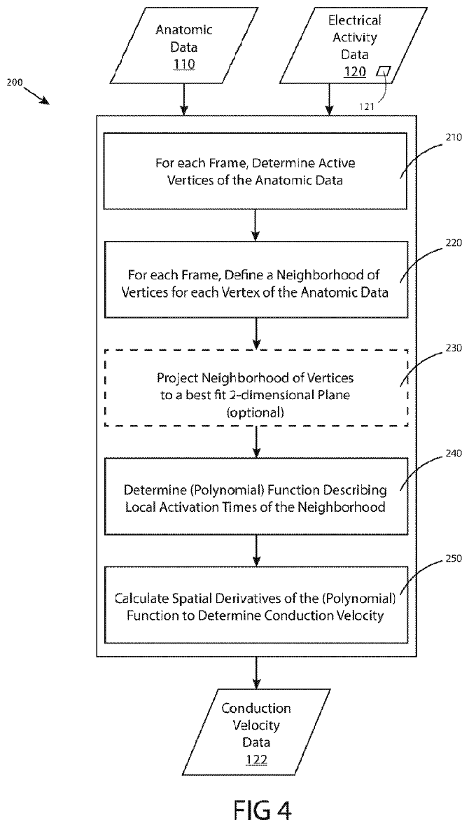

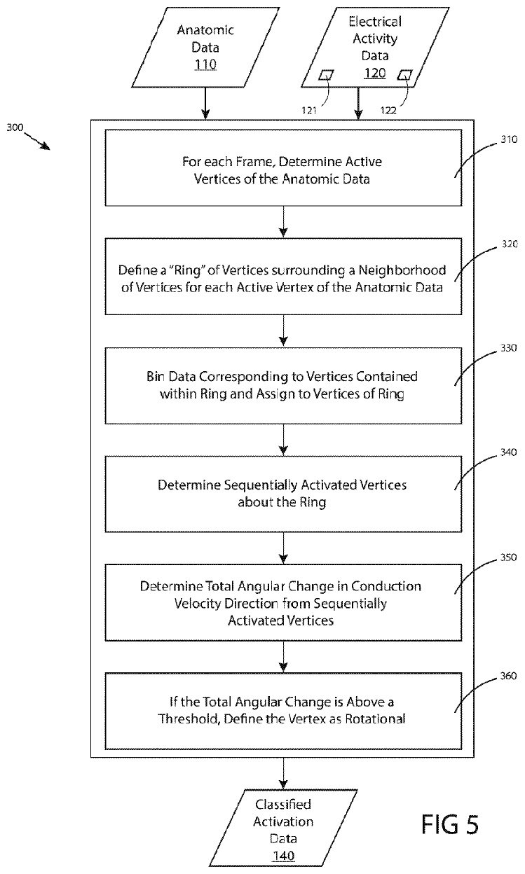

In some embodiments, each frame contains not only the bio-potential data corresponding to each vertex V but also other calculated and/or measured information corresponding to each vertex V. For example, system 100 can include one or more algorithms, as described herebelow, classifying each vertex V for each frame (e.g. classification information that is stored for each frame). Additionally or alternatively, system 100 can “pre-process” recorded bio-potential data, and save the results of the processing for each frame. For example, for each vertex V of each frame, BIO processor 36 can determine if for that instance in time, a vertex is “active” (e.g. along the leading edge of a depolarizing conducting wave propagating through the cardiac tissue), or not. In some embodiments, a binary active or not-active “flag” (i.e. a binary yes/no data point) decreases the processing time for an algorithm. Additionally or alternatively, for each vertex V of each frame, the current activation status and the activation history can be stored (e.g. a history representing if the vertex is active, or had been active within a predetermined time period such as within the previous 100 ms). In these embodiments, the length of the history recorded for each vertex, and/or the resolution of that recording, can be selected (e.g. pre-selected by the manufacturers of system 100, and/or selected by an operator) to balance the speed of one or more algorithms of system 100 versus the overall resolution of the resultant calculations. As used herein, activations “within” a neighborhood can include all activations recorded for each vertex V within the neighborhood for all frames (e.g. for the length of a recording), or it can include only the activations within a time window (e.g. a rolling time window as described herebelow in reference to FIG. 8) of the activation of central vertex CV of the neighborhood, for example within +/−100 ms of the activation of central vertex CV. In some embodiments, an activation is only included in the set of neighborhood activations if the activation is considered within a “minimum and maximum speed estimation”, as described herebelow in reference to FIG. 4. For example, if an activation of a border vertex occurs within 100 ms of the activation of central vertex CV, but the physical distance between the points on the tissue represented by the two vertices is “too long or too short”, such that the computed speed is not within the maximum or minimum speed (e.g. an estimated range of physiological conduction of tissue), the activation is excluded.

In some embodiments, system 100 is constructed and arranged to perform one or more of the algorithms described herein on a portion of mesh 80. For example, a portion of mesh 80 representing tissue proximate the pulmonary veins can be analyzed (e.g. by FA algorithm 500 described herebelow) to identify focal activity, as focal activity near the pulmonary veins has been associated with patients having an arrhythmia such as AF. Additionally or alternatively, one or more algorithms of system 100 can comprise a bias and/or one or more thresholds of an algorithm can be adjusted (e.g. biased) based on the anatomic tissue being analyzed. For example, FA algorithm 500 can be biased towards identifying focal activity proximate the pulmonary veins.

Referring now to FIG. 3, a schematic view of an algorithm for performing a complexity assessment is illustrated, consistent with the present inventive concepts. Algorithm 600 shown can be included in one or more portions of system 100 described hereabove, such as when console 20 comprises algorithm 600. Algorithm 600 is configured to perform a complexity assessment based on recorded bio-potential data, such as bio-potential data recorded by electrodes 12a of catheter 10. Algorithm 600 can perform a complexity assessment based on, as shown in FIG. 3, electrical activity data 120 (e.g. activation timing data 121) and/or anatomic data 110.

In Step 610, for each frame (as described hereabove) the active vertices (also as defined hereabove) of the anatomic data 110 are determined, and activation propagation data is calculated. Step 610 can use an optical flow algorithm (e.g. Horn-Schunck) or other 2D or 3D image-based analysis algorithm to calculate the activation propagation data at each location.

In Step 620, an analysis of the activation propagation data from frame to frame is performed. In this analysis, patterns can be identified, such as rotational patterns, localized irregular patterns, focal activation patterns, and/or other normal or abnormal electrical activity patterns. Patterns can be identified using one or more pattern detection algorithms, such as algorithms 300, 400, and/or 500 described herebelow.

In Step 630, a complexity assessment is performed, such as to produce diagnostic results 1100. Diagnostic results 1100 can be provided to a clinician, such as to determine a therapy to be administered to the patient (e.g. one or more cardiac tissue locations to perform a cardiac ablation procedure, such as using treatment subsystem 800 described hereabove in reference to FIG. 1). In some embodiments, algorithm 600 further includes a complexity algorithm 650 configured to process and/or assess diagnostic results 1100, as described herebelow in reference to FIG. 3A.

Diagnostic results 1100 can comprise scalar values, for example a scalar value assigned to each vertex assessed, representing the “level” of complexity, as calculated over a time period (e.g. time periods TP described herebelow). Additionally or alternatively, diagnostic results 1100 can comprise time varying values, for example a binary value assigned to each vertex assessed, representing “complex” or “not”, calculated for several instances in time (e.g. time period TP1 described herebelow). In some embodiments, binary, time varying values is summed, or otherwise combined, to determine a scalar value of the level of complexity over a longer time period TP (e.g. time period TP2, TP3, or TP4 described herebelow). In some embodiments, binary and/or scalar values are assigned “persistently” to a vertex over subsequent frames of data, for example a binary “yes” can be assigned persistently to a vertex for two, three, or more subsequent frames, potentially overriding a binary “no” from the calculated results. Additionally, repeated positive indicators can be assigned a longer persistence, for example three binary “yes” frames (for a single vertex) can be assigned 5 additional “yes” values (8 total, assuming all relevant subsequent values are “no”), while a single binary “yes” frame can be assigned only 2 additional “yes” values (3 total).

In some embodiments, electrical activity data 120a is recorded (e.g. recorded by electrodes 12a), from at least 10, or at least 48, or at least 64 heart wall locations (e.g. in a contact-mapping procedure). In these embodiments, the vertices determined by system 100 can include the recording locations and/or other heart wall locations. In these embodiments, the electrical activity data can be recorded simultaneously or sequentially.

In some embodiments, electrical activity data 120a is recorded (e.g. recorded by electrodes 12a), from at least 10, or at least 48, or at least 64 locations within a heart chamber (e.g. contacting and/or non-contacting the heart wall). In these embodiments, the vertices determined by system 100 can include the heart-wall based recording locations, and/or other heart wall locations. In these embodiments, the electrical activity data 120 can be recorded simultaneously or sequentially.

Referring additionally to FIG. 3A, complexity algorithm 650 can be configured to process and/or assess diagnostic results 1100 as produced in STEP 630, as described hereabove in reference to FIG. 3. In STEP 6510, algorithm 650 can assess the type and consistency of each complex activation pattern as identified in diagnostic results 1100. In STEPs 6520 and 6530, algorithm 650 can assess the proximity (e.g. in space) and/or the relationship (e.g. in time) between each complex activation pattern, and can then determine if an identified complex activation pattern is part of a “macro-level” complexity activation pattern. In STEP 6540, algorithm 650 can apply a computation method to assess and/or predict a probabilistic outcome of delivering therapy to a location of a macro-level complex activation pattern. In some embodiments, the computational method comprises data analytics/statistics techniques, such as classification or categorization, of electrical activity using a training data set (e.g. separately acquired data, such as historical data) and/or a computationally-optimized fit (e.g. machine learning or predictive analysis, such as by neural network or deep learning, cluster analysis).

STEP 6540 can be configured to provide updated diagnostic results 1100′ as shown, which can include: identification of macro-level complexity; a prioritization of therapeutic targets; a probabilistic and/or predictive therapeutic strategy; one or more modifications to diagnostic results 1100; and combinations of these. In some embodiments, the probabilistic outcome of delivering therapy is determined, or otherwise provided, through the use of machine learning, as described in applicant's co-pending U.S. Patent Provisional Application Ser. No. 62/668,659, titled “CARDIAC INFORMATION PROCESSING SYSTEM”, filed May 8, 2018, the content of which is incorporated herein by reference in its entirety for all purposes. In some embodiments, the predictive therapeutic strategy may be to cause the current rhythm to transition to a less complex rhythm (e.g. to transition from atrial fibrillation to atrial tachycardia), such as a strategy determined using state analysis. The current state of a rhythm can be defined by one or more complexity metrics (e.g. cycle length, number of cardiac waves, Shannon entropy, and/or dominant frequency). State changes can be estimated for various therapeutic strategies (e.g. various ablation locations and/or durations). The therapeutic strategy that is estimated to change the rhythm to the least complex state can then be implemented. Complexity algorithm 650 can take as an input other patient data (e.g. MRI/CT data, patient health history data, and/or previous ablation history data).

Complexity algorithm 600 can comprise an analysis of recorded electrical activity data 120a that is recorded over time periods TP, which can comprise similar or different lengths of time. Each time period TP can represent all or a portion of a continuous recording for that time period TP, or all or a portion of multiple recordings that cumulatively represent the time period TP. In some embodiments, a time period TP represents two or more periods of recording electrical activity as well as the time between recordings. In some embodiments, data that has been recorded over a period of time is segmented into multiple time periods TP (e.g. multiple time periods of the same duration), and a complexity assessment is calculated over each time period TP. The complexity assessment can then be displayed to a user in a video like format (e.g. displayed on display 27a, as described herebelow in reference to FIG. 8). In some embodiments, each time period TP (e.g. time period TP2 described herebelow) comprises a sufficiently long time period TP, such that a user can reasonably perceive the displayed information in a “real rate” fashion (e.g. the information is displayed at the same rate that it occurred). In these embodiments, the displayed information can be presented in a “real time” fashion (e.g. information is displayed as it occurs, with minimal delays due to processing by system 100). Alternatively or additionally, the time period TP can comprise a sufficiently short time period (e.g. time period TP1 described herebelow), such that a user cannot reasonably perceive the displayed information when displayed in a real rate fashion. In these embodiments, a rolling “average” of data can be displayed at a real rate, and/or the data can be replayed in a frame by frame or other slow-motion fashion such that the user can reasonably perceive the data. Additionally or alternatively, various methods of displaying accumulated, summed, averaged, or persistent data can be implemented to provide the user a perceivable time-dependent representation of the calculated data. Furthermore, each time period TP (e.g. TP3 and/or TP4 described herebelow) can comprise an extended time period, and/or a time period spanning two or more discrete recordings, and a time compressed (e.g. time-lapse) data set can be displayed to the user. Playback and other data display modes are described in detail herebelow in reference to FIG. 8.

In some embodiments, a time period TP1 comprises a relatively short time period, such as a period in which between 1-10 activations occur in the cardiac tissue being assessed (e.g. as represented by a set of vertices as described herein). Correspondingly, TP1 can comprise a duration of between 0.3 ms and 2000 ms, such as a time period of approximately 150 ms. In some embodiments, catheter 10 comprises a contact mapping catheter (e.g. a “roving” contact mapping catheter, configured to record electrical activity data 120a via electrodes 12a only from a single discrete portion of the heart chamber at one time). In these embodiments, time period TP1 can approximate the total recording time at a single discrete portion of the heart chamber, a “visit”. A subsequent time period TP1 can approximate a subsequent visit to the same discrete portion of the heart chamber or a different portion. In these embodiments, two, three, or more recordings, each comprising a time period approximately equal to TP1 can be combined to create a more complete data set of recorded electrical activity. The two, three, or more recordings can be combined spatially, based on the portion of the heart chamber recorded, as well as temporally, based on the heart cycle information, as is known in the art of contact cardiac mapping. In some embodiments, catheter 10 comprises a mapping catheter (e.g. a basket catheter), configured to record electrical activity data 120a via electrodes 12a from a distributed set of locations all around the chamber where the electrode locations are intended to be in contact, or near-contact, with the cardiac wall. In some embodiments, catheter 10 comprises a mapping catheter (e.g. a basket catheter), configured to record electrical activity data 120a via electrodes 12a from a distributed set of locations offset with the cardiac wall.

In some embodiments, complexity algorithm 600 comprises an analysis of electrical activity data 120a that is recorded for a time period TP2 that includes a moderate number of electrical activations, such as between 3 and 3000 activations, such as between 10 and 600 activations, or between 25 and 300 activations. Correspondingly, TP2 can comprise a duration of between 0.3 secs and 500 secs, such as a time period between 1 sec and 90 secs or between 4 secs and 30 secs. In some embodiments, time period TP2 represents the length of a single data recording, for example a contact and/or non-contact recording of electrical activity data 120a within a heart chamber.

In some embodiments, complexity algorithm 600 is configured to analyze electrical activity data 120a that is recorded for a time period TP3 that includes a large number of electrical activations, such as between 2,000 and 300,000 activations, such as between 6,000 and 40,000 activations. Correspondingly, TP3 can comprise a duration of between 5 minutes to 8 hours, such as between 15 minutes and 60 minutes. In some embodiments, time period TP3 represents the length of several recordings of acute electrical activity, for example several recordings taken before, after, and/or interspersed between loop-iterations of diagnosis and therapy (e.g. therapy provided by treatment subsystem 800 described hereabove in reference to FIG. 1).

In some embodiments, complexity algorithm 600 is configured to analyze activations and/or electrical data from measurements made with a regional focus. A regional focus can include a region of tissue comprising between approximately 5% and 50% of the heart chamber surface (e.g. between 5% and 50% of the endocardial surface of an atrium or ventricle). The measurements can be made with enough time to capture characteristics of complex conduction representative of the rhythm, such as to capture between approximately 3 and 3000 activations. In some embodiments, electrode array 12 is sequentially maneuvered to different position to form an aggregate map comprising the data from each position.

In some embodiments, complexity algorithm 600 comprises an analysis of electrical activity data 120a that is recorded for a time period TP4 that includes a time period of multiple days, weeks, months, and/or years (e.g. spanning more than one clinical diagnostic procedure performed on the patient). In some embodiments, time period TP4 represents the length of several recordings of electrical activity spanning more than one clinical procedure, for example spanning days, weeks, months, or years.

In some embodiments, complexity algorithm 600 receives additional patient data 150, such as to include both electrical activity data 120 and patient data 150 in a complexity analysis, such as is described hereabove in reference to FIG. 1. In some embodiments, complexity algorithm 600 includes one or more of algorithms 200, 300, 400, and/or 500 described herebelow, each of which can include an assessment of complexity that is based on electrical activity data 120, anatomic data 110, and/or additional patient data 150.

Referring now to FIG. 4, a schematic view of an algorithm for determining conduction velocity data is illustrated, consistent with the present inventive concepts. System 100 can comprise a conduction velocity algorithm, CV algorithm 200, that analyzes anatomic data, data 110 shown, and activation timing data, data 121 shown. Complexity algorithm 600 described hereabove can comprise CV algorithm 200. CV algorithm 200 can comprise one or more instructions executed by a processor of system 100, for example processor 26 of console 20. CV algorithm 200 can process anatomic data 110 and electrical activity data 120 (e.g. activation timing data 121) to determine the conduction velocity at each vertex of the anatomic data 110, for each activation of the associated vertex, as described herein.

In some embodiments, CV algorithm 200 computes one or more components of the velocity (direction and/or magnitude) at each vertex of anatomic data 110 as a depolarizing conducting wave passes through the vertex. The conduction velocity (e.g. the velocity at each vertex as the depolarizing conductive wave passes through the vertex) can be found by determining the spatial gradient of the activation times (t) using the following equation:

$$∇ τ = \frac{dx}{d τ} , \frac{dy}{d τ} , \frac{dz}{d τ} = [ {V}_{x} , {V}_{y} , {V}_{z} ] .$$

Each vertex processed can be considered a “central vertex”, and a small “neighborhood” composed of vertices and activation times proximate each central vertex can be used to estimate the spatial gradient and to find the conduction velocity at the central vertex. In some embodiments, a method for estimating the spatial gradient of activation times for a vertex given a small neighborhood and the positions of the vertices in a small neighborhood comprises fitting the activation times in the neighborhood to a function (e.g. a polynomial function) of the positions of the vertices. In some embodiments, a polynomial surface fitting method is used.

CV algorithm 200 can process each frame of anatomical data 110 and electrical activity data 120a recorded by system 100. In Steps 210-250 described herebelow, processing of a single frame of data is performed. Multiple frames can be processed through the repeating of Steps 210-250 on subsequent frames.

In Step 210, a set of active vertices is determined using anatomic data 110 and electrical activity data 120 (e.g. activation timing data 121).

In Step 220, for each active vertex of the anatomy (for the current frame), a neighborhood of vertices can be defined around that vertex (e.g. a central vertex of that neighborhood). In some embodiments, multiple-edge-connected (e.g. five) neighbors are used to define a neighborhood covering approximately 200 mm²−315 mm² of the anatomical surface with 60-120 vertices included in the neighborhood (for example, neighborhoods as described hereabove in reference to FIG. 2B). Within the neighborhood defined by the multiple-edge-connected neighbors, all activation times t are found that are within a particular minimum speed estimation (e.g. a minimum speed estimation of approximately 0.3 m/s), where speed is estimated as:

$$Speed = \frac{‖ {P}_{center} ( x , y , z ) - {P}_{i} ( x , y , z ) ‖}{\vert {τ}_{center vertex} - {τ}_{i} \vert} ,$$

where P is the position of a vertex.

The principal components of this neighborhood are then determined by creating a matrix of all the vertices positions in the neighbor with the mean removed. Singular value decomposition (SVD) of the matrix of vertex positions can be used to determine the three singular vectors for the local neighborhood, which correspond to the principal components of the neighborhood. The positions of vertices in the neighborhood are transformed into a basis defined by the neighborhood's principal components by multiplying the singular vectors with the positions of each vertex in the neighborhood P_{original}, where:

P_{original}*SingularVectors=Pₚᵣᵢₙᵢₚₐₗ.

After the transform, the neighborhood can be described by spatial variables (uᵢ, vᵢ, kᵢ), where (uᵢ, vᵢ, kᵢ) is the amount of the first, second and third principal component, respectively, used to describe the position of the i^{th} vertex as shown below:

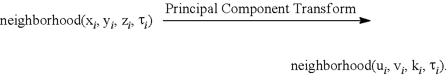

In some embodiments, an optional Step 230 is performed. In Step 230, the singular vector with the smallest singular value in Pₚᵣᵢₙᵢₚₐₗ is removed resulting in converting a 3-dimensional domain to a 2-dimensional planar domain, as performed using the following function:

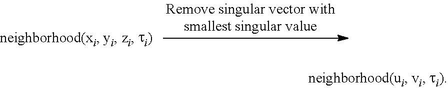

The resulting plane is the best fit plane of the 3-dimensional positions of the vertices converted to a 2-dimensional plane. The 3-dimensional to 2-dimensional transform can be performed to ensure that the computed conduction velocity is tangent to the surface anatomy, and/or to reduce the dimensionality of the polynomial surface fitting performed in subsequent following steps, such as those described herebelow.

In Step 240, a function (e.g. a best fit cubic polynomial surface function, T) is used to describe the local activation times, τᵢ, of the neighborhood as a function of position (uᵢ, vᵢ), for example such that T(uᵢ, vᵢ)≈τᵢ as shown below:

T(u,v)=a₉u³+a₈³+a₇u²v+a₆uv²+a₅u²+a₄v²+a₃uv+a₂u+a₁v+a₀.

Given a set of [u,v]=τ, the following matrix can be constructed to solve for the coefficients A.

$$( \begin{matrix}{u}_{1}^{3} & {v}_{1}^{3} & {u}_{1}^{2} {v}_{1} & {u}_{1} {v}_{1}^{2} & {u}_{1}^{2} & {v}_{1}^{2} & {u}_{1} {v}_{1} & {u}_{1} & {v}_{1} & 1 \\ ⋮ & ⋮ & & ⋮ & ⋮ & ⋮ & ⋮ & ⋮ & ⋮ & ⋮ \\ {u}_{N}^{3} & {v}_{N}^{3} & {u}_{N}^{2} {v}_{N} & {u}_{N} {v}_{N}^{2} & {u}_{N}^{2} & {v}_{N}^{2} & {u}_{N} {v}_{N} & {u}_{N} & {v}_{N} & 1\end{matrix} ) [ \begin{matrix}{a}_{9} \\ ⋮ \\ {a}_{0}\end{matrix} ] = [ \begin{matrix}{τ}_{1} \\ ⋮ \\ {τ}_{n}\end{matrix} ]$$

$$A =$$

The above can be solved with a least squares analysis. Singular value decomposition can be applied to matrix A: A=USV^{T}, from which the pseudo inverse of A can be calculated, which in turn can be used to calculate the coefficients:

[symbol]

=A⁺ [symbol] =(VS⁻¹U^{T})

[symbol]

In Step 250, the conduction velocity can be solved for by analytically finding the derivatives of the surface (e.g. the polynomial surface T), as shown below:

$$= [ \begin{matrix}{V}_{u} \\ {V}_{v}\end{matrix} ] = [ \begin{matrix}\frac{du}{dT} \\ \frac{dv}{dT}\end{matrix} ] = [ \begin{matrix}\frac{\frac{dT}{du}}{{( \frac{dT}{du} )}^{2} + {( \frac{dT}{dv} )}^{2}} \\ \frac{\frac{dT}{dv}}{{( \frac{dT}{du} )}^{2} + {( \frac{dT}{dv} )}^{2}}\end{matrix} ]$$

The conduction velocity can then be normalized to create unit vectors, such as by using the following equation:

$$= \frac{‖ ‖}{}$$

Via the preceding steps, algorithm 200 produces a set of conduction velocity data, data 122 shown, which is based on the anatomic data 110 and activation timing data 121.

In some embodiments, the conduction velocity data 122 can be represented on the anatomical surface (e.g. via display 27a of system 100) by transforming the resulting conduction velocity unit vectors back into the original coordinate system (e.g. the coordinate system of anatomic data 110), such as by using the following equation:

[symbol]

_{u,v Coordinates}*Singular Vectors^{T}= [symbol] _{x,y,z coordinates}.

For each activation (e.g. each activation of each central vertex for each frame), the conduction velocity can be represented 2-dimensionally and/or 3-dimensionally, such as by using the following equation:

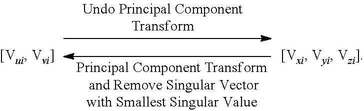

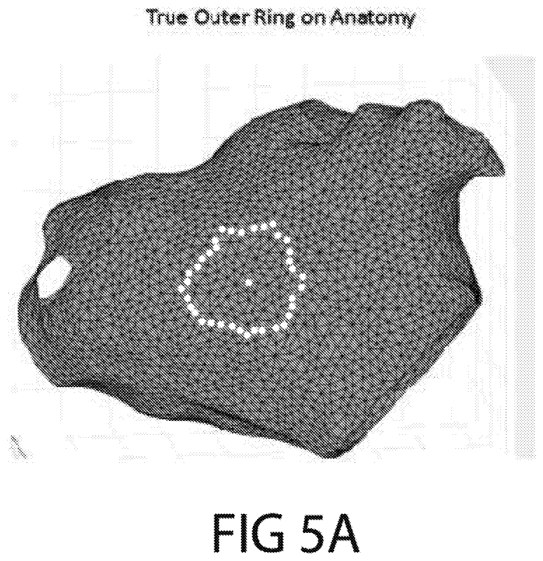

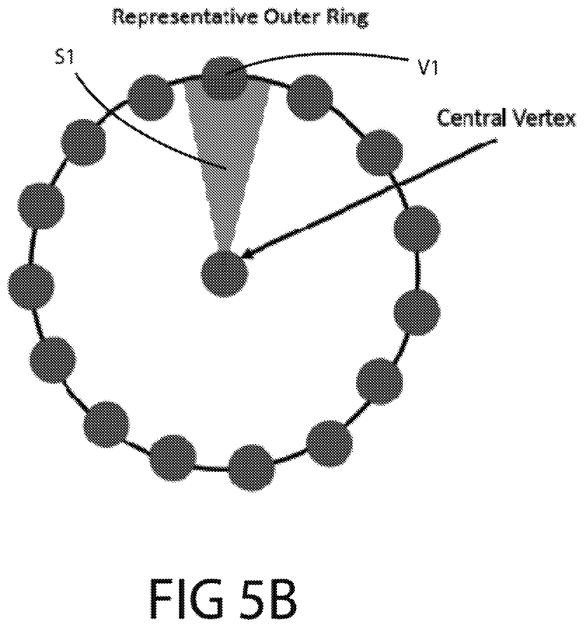

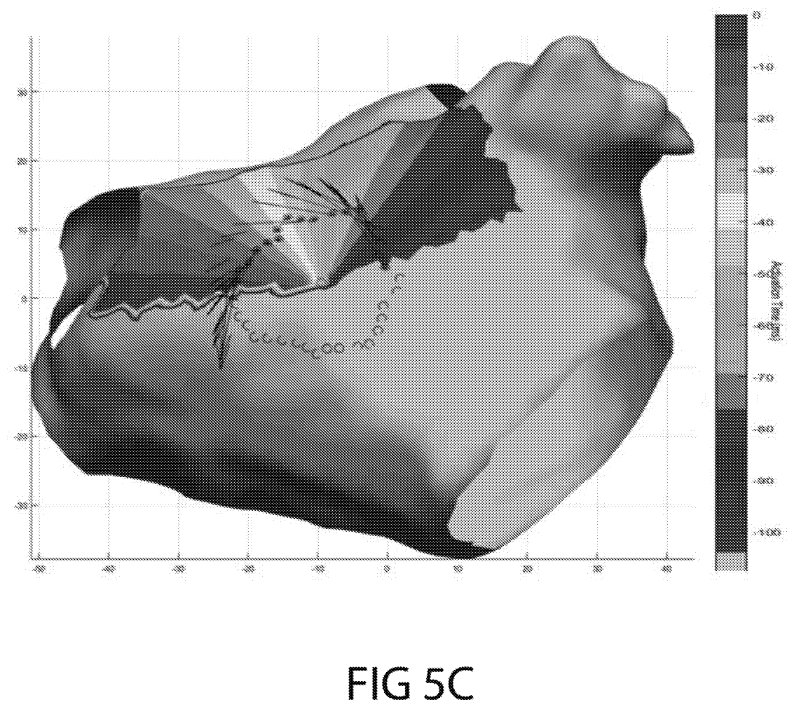

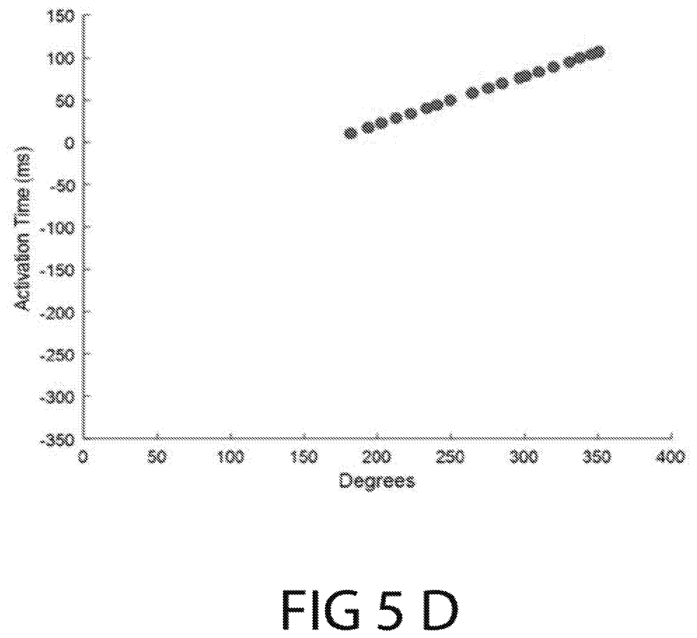

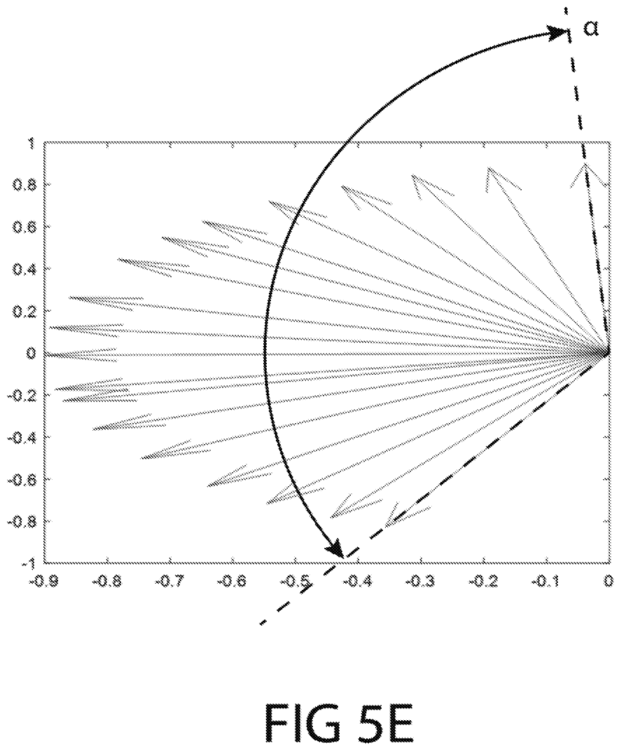

Referring now to FIG. 5, a schematic view of an algorithm for determining localized rotational activity is illustrated, consistent with the present inventive concepts. System 100 can include an algorithm for determining localized rotational activity, LRA algorithm 300. Complexity algorithm 600 described hereabove can comprise LRA algorithm 300. LRA algorithm 300 can be configured to determine the angular change in conduction velocity relative to a central vertex. In atrial fibrillation (AF) and other arrhythmia patients, cardiac electrical activity can manifest as rotors (e.g. rotational electrical activity around a central obstacle). Such rotational activity has long been thought to have a prominent role in the maintenance of a cardiac arrhythmia such as AF (e.g. rotational activity is associated with causing and/or perpetuating these undesired conditions).

In some embodiments, LRA algorithm 300 is used to process each frame of anatomical data 110 and electrical activity data 120 (e.g. activation timing data 121) collected by system 100. In Steps 310-360 described herebelow, processing of a single frame of data is performed. Multiple frames can be processed through the repeating of Steps 310-360 on subsequent frames. In some embodiments, LRA algorithm 300 also includes conduction velocity data 122 in its analysis. Alternatively or additionally, LRA algorithm 300 can be configured to determine conduction velocity data 122, such as when LRA algorithm 300 is configured similar to CV algorithm 200.

In Step 310, a set of active vertices is determined using anatomic data 110 and electrical activation data 120 (e.g. activation timing data 121).

In Step 320, for each active vertex of the anatomy (for the current frame), a neighborhood of vertices can be defined around that vertex (e.g. a central vertex of that neighborhood). For each neighborhood, a ring of vertices around the central vertex can be defined by the boundary vertices of the neighborhood, as shown in FIGS. 5A-B.

In Step 330, for each neighborhood, the activation times and conduction velocities for the vertices in the neighborhood can be grouped (e.g. binned). For each neighborhood, all activation times that are within a particular maximum speed estimation (e.g. a maximum speed estimation of approximately 0.05 m/s) can define (e.g. limit) the set of activations to be grouped. In some embodiments, only activation times that are reachable from a group's center vertex activation with a given maximum speed (e.g. 0.05 m/s), are included within the group. The activations in each neighborhood can be grouped as shown in FIG. 5B. In some embodiments, the average activation timing data 121 and/or the average conduction velocity data 122, for all activations within a group, is assigned to a boundary vertex, also as shown in FIG. 5B.

In Step 340, vertices with a linear trend of activation times (e.g. an increasing or decreasing trend) around the outer ring of vertices are identified. For example, a linear fit with an R2≥0.7 can be identified as a trend. FIG. 5D shows a trend line of activation times.

In Step 350, the total angular change between the average conduction velocities assigned to the first and last vertices of the linear trend identified in Step 340, is determined. FIG. 5E shows the conduction velocities of an identified linear trend that have been translated to an origin point, 0,0. FIG. 5E graphically illustrates the total angular change between average conduction velocities as described hereabove.

In Step 360, LRA algorithm 300 classifies a central vertex as “rotational” if the linear trend identified in Step 340 exceeds a threshold (e.g. an operator-defined threshold) and/or if the total angular change identified in Step 350 exceeds a threshold.

LRA algorithm 300 produces a set of data (e.g. creates new data and/or modifies existing data), classified activation data 140 (e.g. data that has been filtered, categorized, identified and/or otherwise classified to identify activations as being rotational in nature).

Referring now to FIG. 5A, a graphical representation of anatomic data 110 is illustrated, including a neighborhood of vertices defined by an outer ring of vertices.

Referring now to FIG. 5B, a simplified representation of a neighborhood of vertices is illustrated, including an outer ring of vertices positioned about a central vertex. In some embodiments, activations within a neighborhood is segmented, or binned, and subsequently averaged. The average values can be assigned to a single vertex, for example a border vertex within the segment. For example, all activations within an area of the neighborhood represented by shaded portion S1 can be averaged and “assigned” to vertex V1. In some embodiments, the binning is performed to limit the effect of noise on subsequent calculations performed on the data. In some embodiments, the size of segment S1 is chosen to increase the resolution of system 100 (e.g. smaller segments) or to decrease subsequent calculation time (e.g. larger segments).

Referring now to FIG. 5C, a representative anatomy showing an example propagating wave rotating about a neighborhood is illustrated, the neighborhood defined by an outer ring of vertices positioned around a central vertex. Average conduction vectors are also shown from each boundary vertex of the ring.

Referring now to FIG. 5D, a plot of the activation times in the outer ring of vertices of FIG. 5C is illustrated, the activation times plotted against degrees around the central vertex. The points on the plot show a set of vertices in the ring with a linear trend, as described hereabove. In the data shown in FIG. 5D, the trend extends from approximately 200 degrees to approximately 375 degrees, indicative of a cardiac wave that has propagated 175 degrees around the central vertex.

Referring now to FIG. 5E, a graph of conduction velocity vectors associated with FIG. 5C is illustrated, the vectors translated to a point 0,0. The conduction velocity change around the central vertex can be determined by summing up the angles between the sequential conduction velocity vectors. For this example, the conduction velocity vectors of the illustrated data, represented by angle α, sum up to 155 degrees.

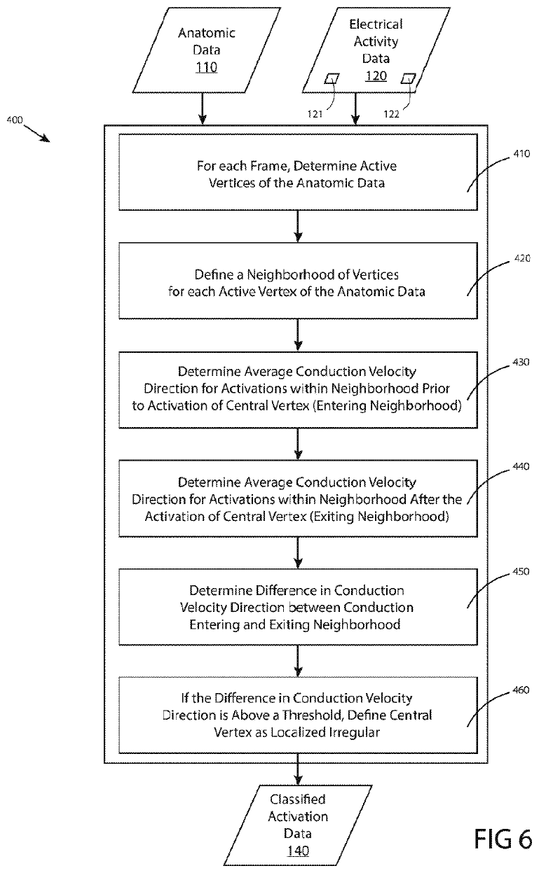

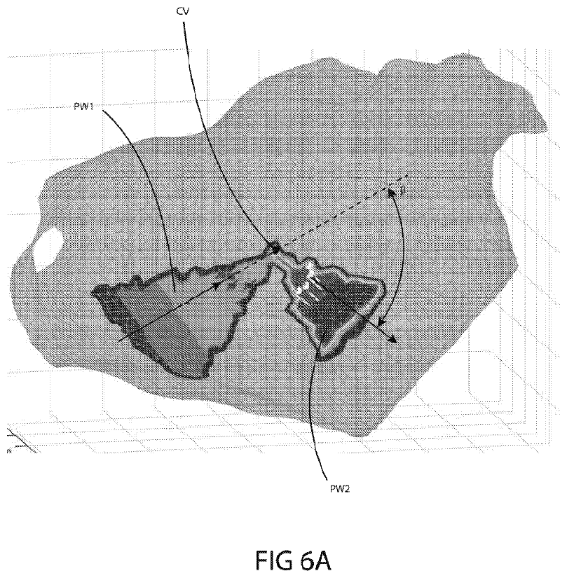

Referring now to FIG. 6, a schematic view of an algorithm for determining localized irregular activity is illustrated, consistent with the present inventive concepts. System 100 can include an algorithm for determining localized irregular activity, LIA algorithm 400. Complexity algorithm 600 described hereabove can comprise LIA algorithm 400. LIA algorithm 400 can be configured to determine the angle between the direction of conduction approaching a central vertex and the direction of conduction departing a central vertex. Irregular activity, such as notable fractionation, irregular reentrant type activity, and/or disorganized conduction, has long been thought to have a prominent role in the maintenance of cardiac arrhythmia, including AF.

In some embodiments, LIA algorithm 400 is used to process each frame of anatomical data 110 and electrical activity data 120 (e.g. activation timing data 121) collected by system 100. In Steps 410-460 described herebelow, processing of a single frame of data is performed. Multiple frames can be processed through the repeating of Steps 410-460 on subsequent frames. In some embodiments, LIA algorithm 400 also includes conduction velocity data 122 in its analysis. Alternatively or additionally, LIA algorithm 400 can be configured to determine conduction velocity data 122, such as when LIA algorithm 400 is configured similar to CV algorithm 200.

In Step 410, a set of active vertices is determined using anatomic data 110 and activation timing data 121.

In Step 420, for each active vertex of the anatomy (for the current frame), a neighborhood of vertices can be defined around that vertex (e.g. a central vertex of that neighborhood). For each neighborhood, a ring of vertices around the central vertex can be defined by the boundary vertices of the neighborhood, such as is shown in FIG. 5A.

In Step 430, for each neighborhood, LIA algorithm 400 can be configured to determine the mean conduction velocity direction for all activations within the neighborhood that: have an earlier activation time than the central vertex's activation time (within a maximum conduction speed, such as a maximum between 0.3 m/s-3 m/s); and have a conduction velocity direction pointing towards the central vertex. In some embodiments, only a subset of these activations is included in the calculation of the mean conduction velocity direction.

In Step 440, for each neighborhood, LIA algorithm 400 can be configured to determine the mean conduction velocity direction for all activations within the neighborhood that: have a later activation time than the central vertex's activation time (within a maximum conduction speed, such as a maximum between 0.3 m/s-3 m/s); and have a conduction velocity direction pointing away from the central vertex. In some embodiments, only a subset of these activations is included in the calculation of the mean conduction velocity direction.

In Step 450, LIA algorithm 400 determines the angle between the mean conduction velocity direction entering the neighborhood, and the mean conduction velocity direction leaving the neighborhood.

In Step 460, LIA algorithm 400 classifies a central vertex as “irregular” if the angle determined in Step 450 exceeds a threshold (e.g. an operator-defined threshold). LIA algorithm 400 produces a set of data (e.g. creates new data and/or modifies existing data), classified activation data 140 (e.g. data that has been filtered, categorized, identified and/or otherwise classified to identify activation as being irregular in nature). In some embodiments, a vertex can be previously classified as rotational (e.g. when LRA algorithm 300 has been performed previously) and LIA algorithm 400 does not reclassify or additionally classify the vertex as irregular. Alternatively or additionally, classified activation data 140 can allow multiple classifications for each vertex. In these embodiments, system 100 can be configured to apply a weighting factor, or otherwise prioritize certain classifications, for example a rotational classification can be considered more important than an irregular classification.

Referring now to FIG. 6A, an example of a propagation wave showing irregular activation is illustrated, consistent with the present inventive concepts. FIG. 6A shows a propagation wave PW1 entering a small region, dot CV. The conduction velocities from PW1 can be averaged to determine a mean conduction velocity direction entering the region CV. FIG. 6A also shows a propagation wave PW2 leaving the region CV. The conduction velocities from PW2 can be averaged to determine a mean conduction velocity direction leaving the region CV. LIA algorithm 400 can be configured to determine the angle β between the direction of conduction approaching CV and the direction of conduction departing CV (as described hereabove). LIA algorithm 400 can classify the central vertex at its activation time as irregular if the angle exceeds a threshold (e.g. a user defined threshold, also as described hereabove).

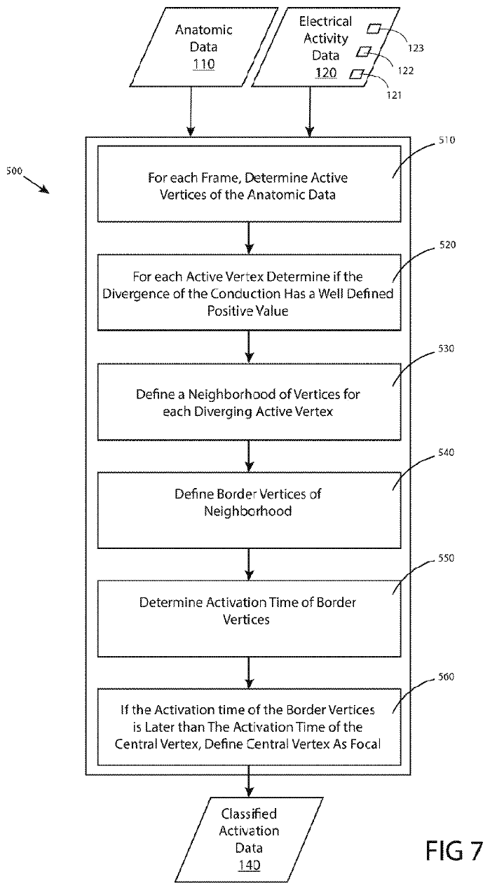

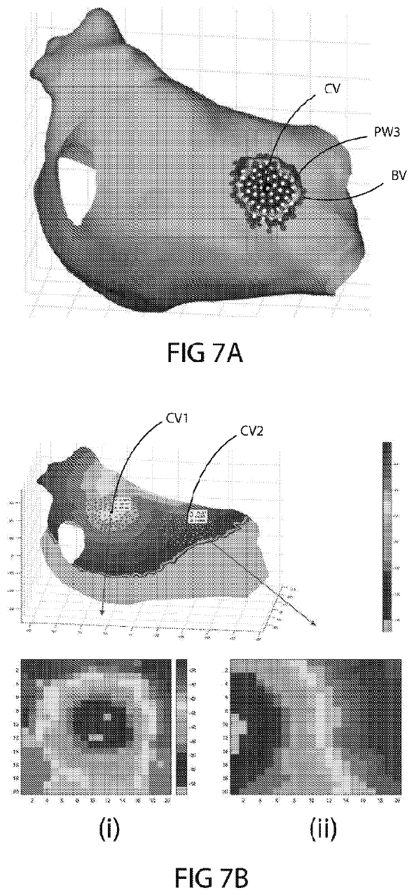

Referring now to FIG. 7, a schematic view of an algorithm for determining focal activation is illustrated, consistent with the present inventive concepts. System 100 can include an algorithm for determining focal activation (also referred to as focal activity), FA algorithm 500. Complexity algorithm 600 described hereabove can comprise FA algorithm 500. FA algorithm 500 can be configured to determine whether an activation at a vertex originated from a previous cardiac wavefront, or whether activation spontaneously started from the vertex (known as focal activation). Focal activation is detected at a vertex if that activation is earlier than the activation of neighboring vertices, and conduction spreads outward from the vertex. Focal activity from the pulmonary veins has been shown to have a pivotal role in maintaining paroxysmal AF. More generally, focal activity is thought to also have a prominent role in the maintenance of cardiac arrhythmia including AF.

In some embodiments, FA algorithm 500 is used to process each frame of anatomical data 110 and electrical activity data 120 (e.g. activation timing data 121) collected by system 100. In Steps 510-560 described herebelow, processing of a single frame of data is performed. Multiple frames can be processed through the repeating of Steps 510-560 on subsequent frames. In some embodiments, FA algorithm 500 also includes conduction velocity data 122 in its analysis. Alternatively or additionally, FA algorithm 500 can be configured to determine conduction velocity data 122, such as when FA algorithm 500 is configured similar to CV algorithm 200. In some embodiments, FA algorithm 500 includes conduction divergence data 123 in its analysis, as defined herebelow. Conduction divergence data 123 can be produced by FA algorithm 500 and/or another algorithm of system 100 (e.g. produced prior to the application of FA algorithm 500).

In some embodiments, conduction divergence data 123 comprises the divergence of conduction velocity from each vertex of anatomic data 110. Divergence of the conduction velocity fields can be defined as:

$$div \vec{V} = \frac{d \vec{v}}{du} + \frac{d \vec{V}}{dv} ,$$

where \vec{V} is the normalized conduction velocity. Similar to the estimation of the conduction velocity, the divergence of the conduction velocities can be estimated by fitting Vᵤ and Vᵥ in a small region to a function (e.g. a 3^{rd} order polynomial) of position, such that,

Vᵤ=F(u,v) and Vᵥ=G(u,v).

The divergence of the vector field can then be computed as:

$$div \vec{V} = \frac{dF}{du} + \frac{dG}{dv} .$$

For every activation of every vertex, if it is determined that the divergence of the conduction velocities has a positive value that exceeds a threshold, the vertex is classified as “well-defined” in conduction divergence data 123. In some embodiments, if half of the vertices within a multiple-edge-connected (e.g. five) neighborhood have a conduction velocity within the minimum conduction velocity range, the divergence is classified as well-defined. A positive divergence threshold of 0.05 can be used.

In Step 510, a set of active vertices is determined using anatomic data 110 and activation timing data 121.

In Step 520, a set of diverging active vertices is identified, from the set of active vertices determined in Step 510.

In Step 530, for each diverging active vertex, a neighborhood of vertices is defined around that vertex (e.g. a central vertex of that neighborhood). For each neighborhood, a ring of vertices around the central vertex can be defined by the boundary vertices of the neighborhood, as shown in FIG. 5A.

In Step 540, a set of “border vertices” is defined, the set containing one-edge-connected neighbors to each boundary vertex of the neighborhood.

In Step 550, the activation time of each border vertex defined in Step 540 is determined.

In Step 560, FA algorithm 500 classifies a central vertex as “focal” if the activation time of each of its border vertices is later than the activation time of the central vertex. FA algorithm 500 produces a set of data (e.g. creates new data and/or modifies existing data), classified activation data 140 (e.g. data that has been filtered, categorized, identified and/or otherwise classified to identify activation as being focal in nature). In some embodiments, a vertex can be previously classified as rotational and/or irregular (e.g. when LRA algorithm 300 and/or LIA algorithm 400 has been performed previously) and FA algorithm 500 does not reclassify or additionally classify the vertex as focal. Alternatively or additionally, classified activation data 140 can allow multiple classifications for each vertex. In these embodiments, system 100 can be configured to apply a weighting factor, or otherwise prioritize certain classifications (e.g. as described hereabove), for example a rotational classification can be considered more important than an irregular and/or focal classification.

Referring now to FIGS. 7A and 7B, a representative anatomy showing focal activation and a representative anatomy showing focal and passive activation are illustrated, respectively, consistent with the present inventive concepts. As shown in FIG. 7A, dot CV shows the current vertex being evaluated. Border vertices BV are shown surrounding a propagation wavefront PW3 that extends from dot CV. As shown in FIG. 7B, dot CV1 shows a first vertex, and dot CV2 shows a second vertex. Zoom window (i) of FIG. 7B shows the neighborhood of vertices about CV1 and zoom window (ii) of FIG. 7B shows the neighborhood of vertices about CV2. In the zoom windows of FIG. 7B, the neighborhoods are shown projected to a plan and interpolated to a regular grid. As described hereabove, complexity algorithm 600 can comprise a supervised learning algorithm, such as a learning algorithm that has been trained on a properly labelled training set. The neighborhood of the central region (e.g. the region about a vertex CV) can be interpolated into a nxm regular grid, such that each value of the grid point contains the activation time, as shown in zoom windows (i) and (ii) in FIG. 7B. Temporal information can be added by concatenating several images together. Once the activation times are on a regular grid, learning algorithms (e.g. feedforward neural networks, convoluted neural networks, and/or support vector machines) can be trained on a large patient set to identify the conduction patterns of interest given an image of the conduction pattern. After the activation time data is evaluated for conduction patterns of interest while transformed into the image space, the labelled output can be put back and displayed (e.g. in 3D anatomical space). In some embodiments, complexity algorithm 600 can be configured to identify electrical patterns selected from the group consisting of: LIA; LRA; focal; slow conduction velocity; isthmus-like conduction; figure of 8's conduction; loop conduction, such as double, triple, or multi-loop conduction; pivoting re-entry; and combinations of these. For example, as shown in zoom (i) of FIG. 7B, focal conduction is illustrated, such as focal conduction that has been identified as a region of interest by algorithm 600. As shown in zoom (ii) of FIG. 7B, passive conduction is illustrated, such as passive conduction that has been identified as a region of “non-interest” by algorithm 600.

Referring now to FIG. 8, an embodiment of a display on which cardiac data (e.g. activation and/or other bio-potential and/or anatomic data) can be rendered is illustrated, consistent with the present inventive concepts. The cardiac data can comprise a series of frames of data that can be dynamically displayed as a function of time. Display 1400 of FIG. 8 can be generated using the same processors, modules, and databases described above for rendering other displays, such as display 27a of FIG. 1. In some embodiments, system 100 and/or display 1400 can be of similar construction and arrangement as displays described in applicant's co-pending International PCT Patent Application Serial Number PCT/US2017/030915, titled “CARDIAC INFORMATION DYNAMIC DISPLAY SYSTEM AND METHOD”, filed May 3, 2017, the content of which is incorporated herein by reference in its entirety for all purposes.

Within a main cardiac information display window or area, window 1405 (e.g. a portion of display 1400), a digital model of cardiac anatomy 1402 is shown with cardiac activation data superimposed or overlaid thereon. In this embodiment, the cardiac activation data is rendered, with an activation status indicated by a series of colors superimposed on the digital cardiac model 1402.

Display 1400 can simultaneously display two or more unique graphical indicia representing different physiological parameters of one or more portions of the heart, as represented by the digital cardiac model 1402 being displayed. The various graphical indicia used to represent these physiologic parameters can be selected from the group consisting of: color; a color range; a pattern; a symbol; a shape; an opacity level; stippling; hue; geometry of a 2D or 3D object; and combinations of these. The graphical indicia used to represent the physiological characteristics can be static and/or dynamic.

The simultaneous display of multiple physiologic characteristics (e.g. as differentiated via the various graphical indicia) can be overlaid on one or more digital models of cardiac anatomy in one or more combinations. Various physiologic parameters, such as minimum re-activation time, conduction velocity, number of occurrences the vorticity threshold was crossed during a time period, and/or other physiologic parameters can each be represented by a unique graphical indicium. A cross-hatch pattern with discrete levels of hatch density and/or line thickness can be overlaid on the digital model, such as to identify regions falling into different categories of conduction velocity. Surface spheroids can be overlaid, centered on nodes with vorticity greater than a threshold, with the diameter of the spheroids displayed proportional to the number of occurrences the vorticity threshold was crossed during the duration of cardiac activity. Hatch patterns and spheroids are provided herein as non-limiting examples of graphical indicia.

In some embodiments, a display of an electrogram, EGM 1410, is presented in an auxiliary cardiac information display window 1415 below the main cardiac information display window 1405 displaying the reconstructed heart 1402.

A set of user-interactive controls, controls 1420, can include a window width control 1422 configured to enable a user to set a time duration for display (e.g. a time duration for which the calculated data displayed represents), in main cardiac information display window 1405, shown here set at 30 ms. The window width (time duration) is indicated in a semitransparent sliding window, window 1412, which is superimposed over EGM 1410. A user-selectable and/or settable display scale, scale 1424, is also provided, which can be used for setting a time scale, t_{SCALE}. Here, t_{SCALE} is set at 3 ms. Accordingly, the horizontal axis of EGM 1410 includes 3 ms increments. Play, rewind, and fast-forward controls, controls 1426, are also included as shown.

In some embodiments, diagnostic results 1100 is displayed in main cardiac information display window 1405, for example a graphic representation of a complexity assessment can be displayed superimposed on reconstructed heart 1402 (e.g. a complexity assessment comprising a calculated value of complexity for each vertex of reconstructed heart 1402). In these embodiments, window width of window 1412 can indicate the portion of recorded data analyzed in the complexity assessment shown (e.g. a time period for which the complexity assessment displayed represents). For example, the displayed complexity assessment can comprise an average of several complexity assessments (calculated over two or more time periods shorter than the within window 1412). The calculation of various complexity assessments is described hereabove. The width of window 1412 can be user selectable and/or adjustable, such as to produce a complexity assessment which includes data from a longer or shorter time period. Two or more complexity assessments can be displayed in a frame by frame fashion (e.g. a movie), where window 1412 “rolls” across EGM 1410 (e.g. a “rolling window”), indicating for each frame what segment of data was analyzed. Alternatively or additionally, a user can position or otherwise adjust window 1412 manually to generate a complexity assessment for a desired segment of the recorded data.

The semitransparent sliding window 1412 is in sync with the cardiac activation data shown overlaid on the reconstructed heart 1402. Therefore, the semitransparent sliding window 1412 and the cardiac activation data overlaid on the reconstructed heart 1402 can dynamically change with respect to a common time scale. The displays are linked in time, and change together, since their outputs are based on the same time-dependent data.

A set of display mode or layer controls, controls 1428, can be provided to enable a user to control at least portions of the display in main window 1405, in particular to control at least portions of the display of cardiac activation data on reconstructed heart 1402. In this embodiment, separate “buttons” (e.g. electro-mechanical switches, touch screen icons, and/or other user-interactive controls) are provided as controls 1428 for selecting “Color Map,” Texture Map,” “Shade Map,” and “Pattern Map” graphical options. In some embodiments, one or more of such controls are provided. Not all such controls need be provided in every embodiment. In some embodiments, none of the controls 1428 need be provided.

In FIG. 8, the reconstructed cardiac chamber 1402 is shown with cardiac activation data represented as varying colors (e.g. varying greyscale, responsive to the Color Map button). For illustration purposes, portions of the reconstructed cardiac chamber 1402 are shown with a texture map 1404 responsive to the Texture Map button, a shade map 1406 responsive to the Shade Map button, and a pattern map 1408 responsive to the Pattern Map button. That is, in some embodiments, such buttons (or similar controls) are used to selectively turn on their respective maps.

For example, a magnitude-indicating graphic (e.g. a graphic indicating, roughness, texture, and the like) which can be uniform, and/or a direction-indicating graphic (e.g. a grain such as a wood grain, line segments, spikes, and the like), which can be directional, can be overlaid on the surface anatomy to visualize conduction or substrate characteristics. A z□ height ‘roughness’ of the magnitude-indicating graphic can be increased or decreased proportionally with the degree of the characteristic displayed (e.g. the magnitude of the characteristic). Also, the direction of block can be shown with a direction-indicating graphic, (e.g. the spikes shown in texture map 1404 of FIG. 8).

Continuing the above example, shading and/or the use of a distinct fixed color palette or gradient (distinct from any other color palette used), such as grayscale, can be used to identify varying degrees of block, such as fixed block, directional block, and/or functional block conditions.

A multi□directional region of activation can be shown with overlays of different unidirectional textures or lines, producing a ‘hatch’ pattern, as shown in pattern map 1408. A calculation of an index of fibrosis and/or other physiologic state index characterizing the surface/substrate can be displayed with a uniform texture, such as a fine pattern, such as a pattern similar in appearance to cement, or a coarser pattern, such as a pattern similar in appearance to pebbles. An index of fibrosis or other physiologic state indices that present an obstruction or obstacle to the conduction pattern can be determined by a combination of velocity, directional uniformity, and/or other conduction pattern characteristics.

Incorporating textures, patterns, shading, and the like, on the surface of the cardiac chamber 1402, provides a way to provide (e.g. visually provide) more information in coordination with other types of cardiac activity information. This configuration is an extended implementation of visual ‘layers’ in the map display that can be used individually or in any combination to provide information related to multiple variables simultaneously, such as through the use of user-interactive controls 1420.

In some embodiments, one or more of the classifications of vertices described herein are indicated on the reconstructed cardiac chamber 1402. In these embodiments, the classification can be indicated as described hereabove, such as with a color overlay and/or other graphical indicia. In some embodiments, colored or otherwise distinguishable “dots” are used to indicate vertices that have been classified as having a particular property (“classified” herein). Overlapping dots and/or other indicators can be used to indicate multiple classifications (e.g. multiple similar and/or different classifications). Overlapping indicators can be displayed in the same location using different radii, height from the surface of the anatomy, and/or offsets along the surface of the anatomy in different directions. In some embodiments, graphic indicators are displayed “persistently”, for example if a vertex is classified in a first frame, an indicator of the classification can persist on the display for one or more subsequent frames. Additionally or alternatively, an indicator of a classification can be displayed for multiple vertices, for example two-edge-connected vertices for a classified vertex.

Referring now to FIGS. 9 and 9A, a schematic view of a mapping catheter, and a perspective anatomic view of a heart chamber with a mapping catheter inserted into the chamber are illustrated, respectively, consistent with the present inventive concepts. Catheter 10′ includes an electrode array 12′, comprising one, two, three or more electrodes 12a. In some embodiments, electrode array 12′ comprises less than 24 electrodes, such as less than 12 electrodes, such as 10, 8, 6, 4, or 3 electrodes. Electrode array 12′ can comprise an expandable array of splines, onto which electrodes 12a are mounted. Catheter 10′ can be percutaneously inserted into a patient, such as to percutaneously deliver electrode array 12′ to a heart chamber (HC), and can be of similar construction and arrangement as catheter 10 described hereabove in reference to FIG. 1. FIG. 9A illustrates electrode array 12′ percutaneously inserted into a heart chamber (HC). Electrodes 12a have been positioned in contact with a portion of the heart wall, such that electrical activity data 120a can be recorded, for example recorded by system 100 as described herein. A region of analysis is illustrated, surrounding the tissue proximate the contact locations of electrodes 12a. In some embodiments, recorded electrical activity data 120a is processed by system 100, for example by performing a complexity analysis using algorithm 600 described hereabove in reference to FIG. 3, and the diagnostic results 1100 generated can be “assigned” to the region of analysis (e.g. the diagnostic results are stored correlating to the vertices of the anatomic model represented within the region of analysis). In some embodiments, diagnostic results 1100 relative to the region of analysis indicate a potential therapeutic benefit from an intervention (e.g. an ablation of tissue) at the region of analysis (e.g. with or without gathering and/or analyzing data from other areas of the heart chamber). In some embodiments, several regions of analysis are interrogated by catheter 10′, for example as electrode array 12′ is repositioned against different portions of the heart chamber (HC) and additional data is recorded and analyzed.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for producing diagnostic results related to a cardiac condition of a patient, comprising:
a diagnostic catheter for insertion into the heart of the patient, the diagnostic catheter configured to record electrical activity data of the patient at multiple recording locations; and
a processing unit for receiving the recorded electrical activity data, and comprising an algorithm configured to:
perform a complexity assessment using the recorded electrical activity data and produce the diagnostic results based on the complexity assessment.

2.-70. (canceled)
